(19) United States
(12) Patent Application Publication
(10) Pub. No.: US 2007/0134764 A1
(43) Pub. Date: Jun. 14, 2007

(54) Endogenous and non-endogenous version of human G protein-coupled receptors

(75) Inventors: Ruoping Chen, San Diego, CA (US); Zhi Liang Chu, San Diego, CA (US); Huong T. Dang, San Diego, CA (US); Kevin P. Lowitz, San Diego, CA (US); Cameron Pride, San Diego, CA (US)

Correspondence Address: BOZICEVIC, FIELD & FRANCIS LLP (AREN);(ARENA PHARMACEUTICALS, INC.), 1900 UNIVERSITY AVENUE, SUITE 200, EAST PALO ALTO, CA, 94303, US

(73) Assignee: Arena Pharmaceuticals, Inc., San Diego, CA (US)

(21) Appl. No.: 11/601,928

(22) Filed: Nov. 20, 2006

Related U.S. Application Data
(63) Continuation of application No. 09/995,225, filed on Nov. 26, 2001.
Application No. 09/995,225 is a continuation-in-part of application No. 09/170,496, filed on Oct. 13, 1998, now Pat. No. 6,555,339.
(60) Provisional application No. 60/253,404, filed on Nov. 27, 2000.
(60) Provisional application No. 60/255,366, filed on Dec. 12, 2000.
(60) Provisional application No. 60/270,286, filed on Feb. 20, 2001.
(60) Provisional application No. 60/282,356, filed on Apr. 6, 2001.
(60) Provisional application No. 60/270,266, filed on Feb. 20, 2001.
(60) Provisional application No. 60/282,032, filed on Apr. 6, 2001.
(60) Provisional application No. 60/282,358, filed on Apr. 6, 2001.
(60) Provisional application No. 60/282,365, filed on Apr. 6, 2001.
(60) Provisional application No. 60/290,917, filed on May 14, 2001.
(60) Provisional application No. 60/309,208, filed on Jul. 31, 2001.

(30) Foreign Application Priority Data
Oct. 12, 1999 (US) PCT/US99/23938

(51) Int. Cl. C07K 14/705; C07H 21/04; C12P 21/06

(52) U.S. Cl. 435/69.1; 536/23.5; 530/350; 435/320.1; 435/325

(57) ABSTRACT

The invention disclosed in this patent document relates to transmembrane receptors, more particularly to a human G protein-coupled receptor for which the endogenous ligand is unknown, and to mutated (non-endogenous) versions of the human GPCRs for evidence of constitutive activity.

### CROSS-REFERENCE TO RELATED APPLICATIONS

This application is a continuation application of U.S. Ser. No. 09/995,225, filed on Nov. 26, 2001, which is continuation-in-part of U.S. Ser. No. 09/170,496, filed Oct. 13, 1998, now U.S. Pat. No. 6,555,339, Issued Apr. 29, 2003, and its corresponding PCT application number PCT/US99/23938, published as WO 00/22129 on Apr. 20, 2000. This document claims the benefit of priority from the following provisional applications, all filed via U.S. Express Mail with the United States Patent and Trademark Office on the indicated dates: U.S. Provisional No. 60/253,404, filed Nov. 27, 2000; U.S. Provisional No. 60/255,366, filed Dec. 12, 2000; U.S. Provisional No. 60/270,286 filed Feb. 20, 2001; U.S. Provisional No. 60/282,356, filed Apr. 6, 2001, which claims priority from U.S. Provisional No. 60/270,266, filed Feb. 20, 2001; U.S. Provisional No. 60/282,032, filed Apr. 6, 2001; U.S. Provisional No. 60/282,358, filed Apr. 6, 2001; U.S. Provisional No. 60/282,365, filed Apr. 6, 2001; U.S. Provisional No. 60/290,917, filed May 14, 2001; U.S. Provisional No. 60/309,208, filed Jul. 31, 2001. The disclosures of each of the foregoing are hereby incorporated in their entirety by reference.

### FIELD OF THE INVENTION

The present invention relates to transmembrane receptors, in some embodiments to G protein-coupled receptors and, in some preferred embodiments, to endogenous GPCRs that are altered to establish or enhance constitutive activity of the receptor. In some embodiments, the constitutively activated GPCRs will be used for the direct identification of candidate compounds as receptor agonists or inverse agonists having applicability as therapeutic agents.

### BACKGROUND OF THE INVENTION

Although a number of receptor classes exist in humans, by far the most abundant and therapeutically relevant is represented by the G protein-coupled receptor (GPCR) class. It is estimated that there are some 30,000-40,000 genes within the human genome, and of these, approximately 2% are estimated to code for GPCRs. Receptors, including GPCRs, for which the endogenous ligand has been identified, are referred to as “known” receptors, while receptors for which the endogenous ligand has not been identified are referred to as “orphan” receptors.

GPCRs represent an important area for the development of pharmaceutical products: from approximately 20 of the 100 known GPCRs, approximately 60% of all prescription pharmaceuticals have been developed. For example, in 1999, of the top 100 brand name prescription drugs, the following drugs interact with GPCRs (diseases and/or disorders treated are indicated in parentheses):

| | | |
|---|---|---|
| Claritin ® (allergies) | Prozac ® (depression) | Vasotec ® (hypertension) |
| Paxil ® (depression) | Zoloft ® (depression) | Zyprexa ® (psychotic disorder) |
| Cozaar ® (hypertension) | Imitrex ® (migraine) | Zantac ® (reflux) |
| Propulsid ® (reflux disease) | Risperdal ® (schizophrenia) | Serevent ® (asthma) |
| Pepcid ® (reflux) | Gaster ® (ulcers) | Atrovent ® (bronchospasm) |
| Effexor ® (depression) | Depakote ® (epilepsy) | Cardura ® (prostatic hypertrophy) |
| Allegra ® (allergies) | Lupron ® (prostate cancer) | Zoladex ® (prostate cancer) |
| Diprivan ® (anesthesia) | BuSpar ® (anxiety) | Ventolin ® (bronchospasm) |
| Hytrin ® (hypertension) | Wellbutrin ® (depression) | Zyrtec ® (rhinitis) |
| Plavix ® (MI/stroke) | Toprol-XL ® (hypertension) | Tenormin ® (angina) |
| Xalatan ® (glaucoma) | Singulair ® (asthma) | Diovan ® (hypertension) |
| Harnal ® (prostatic hyperplasia) | | |

(Med Ad News 1999 Data).

GPCRs share a common structural motif, having seven sequences of between 22 to 24 hydrophobic amino acids that form seven alpha helices, each of which spans the membrane (each span is identified by number, i.e., transmembrane-1 (TM-1), transmebrane-2 (TM-2), etc.). The transmembrane helices are joined by strands of amino acids between transmembrane-2 and transmembrane-3, transmembrane-4 and transmembrane-5, and transmembrane-6 and transmembrane-7 on the exterior, or “extracellular” side, of the cell membrane (these are referred to as “extracellular” regions 1, 2 and 3 (EC-1, EC-2 and EC-3), respectively). The transmembrane helices are also joined by strands of amino acids between transmembrane-1 and transmembrane-2, transmembrane-3 and transmembrane-4, and transmembrane-5 and transmembrane-6 on the interior, or “intracellular” side, of the cell membrane (these are referred to as “intracellular” regions 1, 2 and 3 (IC-1, IC-2 and IC-3), respectively). The “carboxy” (“C”) terminus of the receptor lies in the intracellular space within the cell, and the “amino” (“N”) terminus of the receptor lies in the extracellular space outside of the cell.

Generally, when an endogenous ligand binds with the receptor (often referred to as “activation” of the receptor), there is a change in the conformation of the intracellular region that allows for coupling between the intracellular region and an intracellular “G-protein.” It has been reported that GPCRs are “promiscuous” with respect to G proteins, i.e., that a GPCR can interact with more than one G protein. See, Kenakin, T., 43 Life Sciences 1095 (1988). Although other G proteins exist, currently, G_{q}, Gₛ, Gᵢ, G_{z} and Gₒ are G proteins that have been identified. Ligand-activated GPCR coupling with the G-protein initiates a signaling cascade process (referred to as “signal transduction”). Under normal conditions, signal transduction ultimately results in cellular activation or cellular inhibition. Although not wishing to be bound to theory, it is thought that the IC-3 loop as well as the carboxy terminus of the receptor interact with the G protein.

Under physiological conditions, GPCRs exist in the cell membrane in equilibrium between two different conformations: an “inactive” state and an “active” state. A receptor in an inactive state is unable to link to the intracellular signaling transduction pathway to initiate signal transduction leading to a biological response. Changing the receptor conformation to the active state allows linkage to the transduction pathway (via the G-protein) and produces a biological response.

A receptor may be stabilized in an active state by a ligand or a compound such as a drug. Recent discoveries, including but not exclusively limited to modifications to the amino acid sequence of the receptor, provide means other than ligands or drugs to promote and stabilize the receptor in the active state conformation. These means effectively stabilize the receptor in an active state by simulating the effect of a ligand binding to the receptor. Stabilization by such ligand-independent means is termed “constitutive receptor activation.”

### SUMMARY OF THE INVENTION

Disclosed herein are endogenous and non-endogenous versions of human GPCRs and uses thereof.

Some embodiments of the present invention relate to a G protein-coupled receptor encoded by an amino acid sequence of SEQ. ID. NO.:2, non-endogenous, constitutively activated versions of the same, and host cells comprising the same.

Some embodiments of the present invention relate to a plasmid comprising a vector and the cDNA of SEQ. ID. NO.:1 and host cells comprising the same.

Some embodiments of the present invention relate to a G protein-coupled receptor encoded by an amino acid sequence of SEQ. ID. NO.:4, non-endogenous, constitutively activated versions of the same, and host cells comprising the same.

Some embodiments of the present invention relate to a plasmid comprising a vector and the cDNA of SEQ. ID. NO.:3, non-endogenous, constitutively activated versions of the same, and host cells comprising the same.

Some embodiments of the present invention relate to G protein-coupled receptor encoded by an amino acid sequence of SEQ. ID. NO.:6, non-endogenous, constitutively activated versions of the same, and host cells comprising the same.

Some embodiments of the present invention relate to a plasmid comprising a vector and the cDNA of SEQ. ID. NO.:5 and host cells comprising the same.

Some embodiments of the present invention relate to a G protein-coupled receptor encoded by an amino acid sequence of SEQ. ID. NO.:8, non-endogenous, constitutively activated versions of the same, and host cells comprising the same.

Some embodiments of the present invention relate to a plasmid comprising a vector and the cDNA of SEQ. ID. NO.:7, non-endogenous, constitutively activated versions of the same, and host cells comprising the same.

Some embodiments of the present invention relate to a G protein-coupled receptor encoded by an amino acid sequence of SEQ. ID. NO.:10, non-endogenous, constitutively activated versions of the same, and host cells comprising the same.

Some embodiments of the present invention relate to a plasmid comprising a vector and the cDNA of SEQ. ID. NO.:9 and host cells comprising the same.

Some embodiments of the present invention relate to a G protein-coupled receptor encoded by an amino acid sequence of SEQ. ID. NO.:12, non-endogenous, constitutively activated versions of the same, and host cells comprising the same.

Some embodiments of the present invention relate to a plasmid comprising a vector and the cDNA of SEQ. ID. NO.:1, and host cells comprising the same.

Some embodiments of the present invention relate to a G protein-coupled receptor encoded by an amino acid sequence of SEQ. ID. NO.:14, constitutively activated versions of the same, and host cells comprising the same.

Some embodiments of the present invention relate to a plasmid comprising a vector and the cDNA of SEQ. ID. NO.:13 and host cells comprising the same.

Some embodiments of the present invention relate to a G protein-coupled receptor encoded by an amino acid sequence of SEQ. ID. NO.:16, constitutively activated versions of the same, and host cells comprising the same.

Some embodiments of the present invention relate to a plasmid comprising a vector and the cDNA of SEQ. ID. NO.:15 and host cells comprising the same.

Some embodiments of the present invention relate to a G protein-coupled receptor encoded by an amino acid sequence of SEQ. ID. NO.:18, constitutively activated versions of the same, and host cells comprising the same.

Some embodiments of the present invention relate to a plasmid comprising a vector and the cDNA of SEQ. ID. NO.:17 and host cells comprising the same.

Some embodiments of the present invention relate to a G protein-coupled receptor encoded by an amino acid sequence of SEQ. ID. NO.:20, constitutively activated versions of the same, and host cells comprising the same.

Some embodiments of the present invention relate to a plasmid comprising a vector and the cDNA of SEQ. ID. NO.:19 and host cells comprising the same.

### BRIEF DESCRIPTION OF THE DRAWINGS

FIG. 1 is a graphic representation of activation of RUP32, G_{q}(del)/Gᵢ, RUP32 co-transfected with G_{q}(del)/Gᵢ, and CMV (control; expression vector) in a second messenger assay measuring the accumulation of inositol phosphate (IP₃) utilizing 293 cells.

FIG. 2 provides an illustration of second messenger IP₃ production from endogenous version RUP35 and RUP36 as compared with the control (“CMV”).

### DETAILED DESCRIPTION

The scientific literature that has evolved around receptors has adopted a number 4 of terms to refer to ligands having various effects on receptors. For clarity and consistency, the following definitions will be used throughout this patent document. To the extent that these definitions conflict with other definitions for these terms, the following definitions shall control:

AGONISTS shall mean materials (e.g., ligands, candidate compounds) that activate the intracellular response when they bind to the receptor, or enhance GTP binding to membranes. In some embodiments, AGONISTS are those materials not previously known to activate the intracellular response when they bind to the receptor or to enhance GTP binding to membranes.

AMINO ACID ABBREVIATIONS used herein are set out in Table A:

TABLE A

| | | |
|---|---|---|
| ALANINE | ALA | A |
| ARGININE | ARG | R |
| ASPARAGINE | ASN | N |
| ASPARTIC ACID | ASP | D |
| CYSTEINE | CYS | C |
| GLUTAMIC ACID | GLU | E |
| GLUTAMINE | GLN | Q |
| GLYCINE | GLY | G |
| HISTIDINE | HIS | H |
| ISOLEUCINE | ILE | I |
| LEUCINE | LEU | L |
| LYSINE | LYS | K |
| METHIONINE | MET | M |
| PHENYLALANINE | PHE | F |
| PROLINE | PRO | P |
| SERINE | SER | S |
| THREONINE | THR | T |
| TRYPTOPHAN | TRP | W |
| TYROSINE | TYR | Y |
| VALINE | VAL | V |

ANTAGONIST shall mean materials (e.g., ligands, candidate compounds) that competitively bind to the receptor at the same site as the agonists but which do not activate the intracellular response initiated by the active form of the receptor, and can thereby inhibit the intracellular responses by agonists. ANTAGONISTS do not diminish the baseline intracellular response in the absence of an agonist. In some embodiments, ANTAGONISTS are those materials not previously known to activate the intracellular response when they bind to the receptor or to enhance GTP binding to membranes.

CANDIDATE COMPOUND shall mean a molecule (for example, and not limitation, a chemical compound) that is amenable to a screening technique. Preferably, the phrase “candidate compound” does not include compounds which were publicly known to be compounds selected from the group consisting of inverse agonist, agonist or antagonist to a receptor, as previously determined by an indirect identification process (“indirectly identified compound”); more preferably, not including an indirectly identified compound which has previously been determined to have therapeutic efficacy in at least one mammal; and, most preferably, not including an indirectly identified compound which has previously been determined to have therapeutic utility in humans.

COMPOSITION means a material comprising at least one component; a “pharmaceutical composition” is an example of a composition.

COMPOUND EFFICACY shall mean a measurement of the ability of a compound to inhibit or stimulate receptor functionality; i.e. the ability to activate/inhibit a signal transduction pathway, as opposed to receptor binding affinity. Exemplary means of detecting compound efficacy are disclosed in the Example section of this patent document.

CODON shall mean a grouping of three nucleotides (or equivalents to nucleotides) which generally comprise a nucleoside (adenosine (A), guanosine (G), cytidine (C), uridine (U) and thymidine (T)) coupled to a phosphate group and which, when translated, encodes an amino acid.

CONSTITUTIVELY ACTIVATED RECEPTOR shall mean a receptor subjected to constitutive receptor activation. A constitutively activated receptor can be endogenous or non-endogenous.

CONSTITUTIVE RECEPTOR ACTIVATION shall mean stabilization of a receptor in the active state by means other than binding of the receptor with its ligand or a chemical equivalent thereof.

CONTACT or CONTACTING shall mean bringing at least two moieties together, whether in an in vitro system or an in vivo system.

DIRECTLY IDENTIFYING or DIRECTLY IDENTIFIED, in relationship to the phrase “candidate compound”, shall mean the screening of a candidate compound against a constitutively activated receptor, preferably a constitutively activated orphan receptor, and most preferably against a constitutively activated G protein-coupled cell surface orphan receptor, and assessing the compound efficacy of such compound. This phrase is, under no circumstances, to be interpreted or understood to be encompassed by or to encompass the phrase “indirectly identifying” or “indirectly identified.”

ENDOGENOUS shall mean a material that a mammal naturally produces. ENDOGENOUS in reference to, for example and not limitation, the term “receptor,” shall mean that which is naturally produced by a mammal (for example, and not limitation, a human) or a virus. By contrast, the term NON-ENDOGENOUS in this context shall mean that which is not naturally produced by a mammal (for example, and not limitation, a human) or a virus. For example, and not limitation, a receptor which is not constitutively active in its endogenous form, but when manipulated becomes constitutively active, is most preferably referred to herein as a “non-endogenous, constitutively activated receptor.” Both terms can be utilized to describe both “in vivo” and “in vitro” systems. For example, and not limitation, in a screening approach, the endogenous or non-endogenous receptor may be in reference to an in vitro screening system. As a further example and not limitation, where the genome of a mammal has been manipulated to include a non-endogenous constitutively activated receptor, screening of a candidate compound by means of an in vivo system is viable.

G PROTEIN COUPLED RECEPTOR FUSION PROTEIN and GPCR FUSION PROTEIN, in the context of the invention disclosed herein, each mean a non-endogenous protein comprising an endogenous, constitutively activate GPCR or a non-endogenous, constitutively activated GPCR fused to at least one G protein, most preferably the alpha (α) subunit of such G protein (this being the subunit that binds GTP), with the G protein preferably being of the same type as the G protein that naturally couples with endogenous orphan GPCR. For example, and not limitation, in an endogenous state, if the G protein “Gₛα” is the predominate G protein that couples with the GPCR, a GPCR Fusion Protein based upon the specific GPCR would be a non-endogenous protein comprising the GPCR fused to Gₛα; in some circumstances, as will be set forth below, a non-predominant G protein can be fused to the GPCR. The G protein can be fused directly to the C-terminus of the constitutively active GPCR or there may be spacers between the two.

HOST CELL shall mean a cell capable of having a Plasmid and/or Vector incorporated therein. In the case of a prokaryotic Host Cell, a Plasmid is typically replicated as a autonomous molecule as the Host Cell replicates (generally, the Plasmid is thereafter isolated for introduction into a eukaryotic Host Cell); in the case of a eukaryotic Host Cell, a Plasmid is integrated into the cellular DNA of the Host Cell such that when the eukaryotic Host Cell replicates, the Plasmid replicates. In some embodiments the Host Cell is eukaryotic, more preferably, mammalian, and most preferably selected from the group consisting of 293, 293T and COS-7 cells.

INDIRECTLY IDENTIFYING or INDIRECTLY IDENTIFIED means the traditional approach to the drug discovery process involving identification of an endogenous ligand specific for an endogenous receptor, screening of candidate compounds against the receptor for determination of those which interfere and/or compete with the ligand-receptor interaction, and assessing the efficacy of the compound for affecting at least one second messenger pathway associated with the activated receptor.

INHIBIT or INHIBITING, in relationship to the term “response” shall mean that a response is decreased or prevented in the presence of a compound as opposed to in the absence of the compound.

INVERSE AGONISTS shall mean materials (e.g., ligand, candidate compound) which bind to either the endogenous form of the receptor or to the constitutively activated form of the receptor, and which inhibit the baseline intracellular response initiated by the active form of the receptor below the normal base level of activity which is observed in the absence of agonists, or decrease GTP binding to membranes. Preferably, the baseline intracellular response is inhibited in the presence of the inverse agonist by at least 30%, at least 50%, at least 60%, at least 70%, at least 75%, at least 80%, at least 85%, at least 90%, at least 92%, at least 94%, at least 95%, at least 96%, at least 97%, at least 98%, and most preferably at least 99% as compared with the baseline response in the absence of the inverse agonist.

KNOWN RECEPTOR shall mean an endogenous receptor for which the endogenous ligand specific for that receptor has been identified.

LIGAND shall mean a molecule specific for a naturally occurring receptor.

MUTANT or MUTATION in reference to an endogenous receptor's nucleic acid and/or amino acid sequence shall mean a specified change or changes to such endogenous sequences such that a mutated form of an endogenous, non-constitutively activated receptor evidences constitutive activation of the receptor. In terms of equivalents to specific sequences, a subsequent mutated form of a human receptor is considered to be equivalent to a first mutation of the human receptor if (a) the level of constitutive activation of the subsequent mutated form of a human receptor is substantially the same as that evidenced by the first mutation of the receptor; and (b) the percent sequence (amino acid and/or nucleic acid) homology between the subsequent mutated form of the receptor and the first mutation of the receptor is at least 80%, at least 85%, at least 90%, at least 92%, at least 94%, at least 95%, at least 96%, at least 97%, at least 98%, and most preferably at least 99%. In some embodiments, owing to the fact that some preferred cassettes disclosed herein for achieving constitutive activation include a single amino acid and/or codon change between the endogenous and the non-endogenous forms of the GPCR, it is preferred that the percent sequence homology should be at least 98%.

NON-ORPHAN RECEPTOR shall mean an endogenous naturally occurring molecule specific for an identified ligand wherein the binding of a ligand to a receptor activates an intracellular signaling pathway.

ORPHAN RECEPTOR shall mean an endogenous receptor for which the ligand specific for that receptor has not been identified or is not known.

PHARMACEUTICAL COMPOSITION shall mean a composition comprising at least one active ingredient, whereby the composition is amenable to investigation for a specified, efficacious outcome in a mammal (for example, and not limitation, a human). Those of ordinary skill in the art will understand and appreciate the techniques appropriate for determining whether an active ingredient has a desired efficacious outcome based upon the needs of the artisan.

PLASMID shall mean the combination of a Vector and cDNA. Generally, a Plasmid is introduced into a Host Cell for the purposes of replication and/or expression of the cDNA as a protein.

SECOND MESSENGER shall mean an intracellular response produced as a result of receptor activation. A second messenger can include, for example, inositol triphosphate (IP₃), diacycglycerol (DAG), cyclic AMP (cAMP), and cyclic GMP (cGMP). Second messenger response can be measured for a determination of receptor activation. In addition, second messenger response can be measured for the direct identification of candidate compounds, including for example, inverse agonists, agonists, and antagonists.

SIGNAL TO NOISE RATIO shall mean the signal generated in response to activation, amplification, or stimulation wherein the signal is above the background noise or the basal level in response to non-activation, non-amplification, or non-stimulation.

SPACER shall mean a translated number of amino acids that are located after the last codon or last amino acid of a gene, for example a GPCR of interest, but before the start codon or beginning regions of the G protein of interest, wherein the translated number amino acids are placed in-frame with the beginnings regions of the G protein of interest. The number of translated amino acids can be tailored according to the needs of the skilled artisan and is generally from about one amino acid, preferably two amino acids, more preferably three amino acids, more preferably four amino acids, more preferably five amino acids, more preferably six amino acids, more preferably seven amino acids, more preferably eight amino acids, more preferably nine amino acids, more preferably ten amino acids, more preferably eleven amino acids, and even more preferably twelve amino acids.

STIMULATE or STIMULATING, in relationship to the term “response” shall mean that a response is increased in the presence of a compound as opposed to in the absence of the compound.

SUBSTANTIALLY shall refer to a result which is within 40% of a control result, preferably within 35%, more preferably within 30%, more preferably within 25%, more preferably within 20%, more preferably within 15%, more preferably within 10%, more preferably within 5%, more preferably within 2%, and most preferably within 1% of a control result. For example, in the context of receptor functionality, a test receptor may exhibit substantially similar results to a control receptor if the transduced signal, measured using a method taught herein or similar method known to the art-skilled, if within 40% of the signal produced by a control signal.

VECTOR in reference to cDNA shall mean a circular DNA capable of incorporating at least one cDNA and capable of incorporation into a Host Cell.

The order of the following sections is set forth for presentational efficiency and is not intended, nor should be construed, as a limitation on the disclosure or the claims to follow.

A. Introduction

The traditional study of receptors has typically proceeded from the a priori assumption (historically based) that the endogenous ligand must first be identified before discovery could proceed to find antagonists and other molecules that could affect the receptor. Even in cases where an antagonist might have been known first, the search immediately extended to looking for the endogenous ligand. This mode of thinking has persisted in receptor research even after the discovery of constitutively activated receptors. What has not been heretofore recognized is that it is the active state of the receptor that is most useful for discovering agonists and inverse agonists of the receptor. For those diseases which result from an overly active receptor or an under-active receptor, what is desired in a therapeutic drug is a compound which acts to diminish the active state of a receptor or enhance the activity of the receptor, respectively, not necessarily a drug which is an antagonist to the endogenous ligand. This is because a compound that reduces or enhances the activity of the active receptor state need not bind at the same site as the endogenous ligand. Thus, as taught by a method of this invention, any search for therapeutic compounds should start by screening compounds against the ligand-independent active state.

B. Identification of Human GPCRs

The efforts of the Human Genome project has led to the identification of a plethora of information regarding nucleic acid sequences located within the human genome; it has been the case in this endeavor that genetic sequence information has been made available without an understanding or recognition as to whether or not any particular genomic sequence does or may contain open-reading frame information that translate human proteins. Several methods of identifying nucleic acid sequences within the human genome are within the purview of those having ordinary skill in the art. For example, and not limitation, a variety of human GPCRs, disclosed herein, were discovered by reviewing the GenBank™ database. Table B, below, lists several endogenous GPCRs that we have discovered, along with other GPCRs that are homologous to the disclosed GPCR.

TABLE B

| Disclosed | | | | | Per Cent |
|---|---|---|---|---|---|
| Human | Accession | Open Reading | | Reference To | Homology |
| Orphan | Number | Frame | | Homologous | To Designated |
| GPCRs | Identified | (Base Pairs) | | GPCR | GPCR |
| hRUP28 | AC073957 | 1,002 | bp | hGPR30 | 34% |
| hRUP29 | AC083865 | 918 | bp | hGPR18 | 27% |
| hRUP30 | AC055863 | 1,125 | bp | hBRB1 | 27% |
| hRUP31 | AL356214 | 1,086 | bp | hGALR-1 | 31% |
| hRUP32 | AL513524 | 1,038 | bp | hPNR | 43% |
| hRUP33 | AL513524 | 1,020 | bp | GPR57 | 50% |
| | | | | GPR58 | 51% |
| hRUP34 | AL513524 | 1,029 | bp | hPNR | 45% |
| hRUP35 | AC021089 | 1,062 | bp | hκ-type 3 | 27% |
| | | | | opioid | |
| hRUP36 | AC090099 | 969 | bp | GPR90 | 42% |
| hRUP37 | AC090099 | 969 | bp | hMRG | 41% |

Receptor homology is useful in terms of gaining an appreciation of a role of the receptors within the human body. As the patent document progresses, techniques for mutating these receptors to establish non-endogenous, constitutively activated versions of these receptors will be discussed.

The techniques disclosed herein are also applicable to other human GPCRs known to the art, as will be apparent to those skilled in the art.

C. Receptor Screening

Screening candidate compounds against a non-endogenous, constitutively activated version of the GPCRs disclosed herein allows for the direct identification of candidate compounds which act at the cell surface receptor, without requiring use of the receptor's endogenous ligand. Using routine, and often commercially available techniques, one can determine areas within the body where the endogenous version of human GPCRs disclosed herein is expressed and/or over-expressed. The expression location of a receptor in a specific tissue provides a scientist with the ability to assign a physiological functional role of the receptor. It is also possible using these techniques to determine related disease/disorder states which are associated with the expression and/or over-expression of the receptor; such an approach is disclosed in this patent document. Furthermore, expression of a receptor in diseased organs can assist one in determining the magnitude of the clinical relevance of the receptor.

Constitutive activation of the GPCRs disclosed herein is based upon the distance from the proline residue at which is presumed to be located within TM6 of the GPCR; this algorithmic technique is disclosed in co-pending and commonly assigned patent document PCT Application Number PCT/US99/23938, published as WO 00/22129 on Apr. 20, 2000, which, along with the other patent documents listed herein, is incorporated herein by reference. The algorithmic technique is not predicated upon traditional sequence “alignment” but rather a specified distance from the aforementioned TM6 proline residue (or, of course, endogenous constitutive substitution for such proline residue). By mutating the amino acid residue located 16 amino acid residues from this residue (presumably located in the IC3 region of the receptor) to, most preferably, a lysine residue, constitutive activation of the receptor may be obtained. Other amino acid residues may be useful in the mutation at this position to achieve this objective and will be discussed in detail, below.

D. Disease/Disorder Identification and/or Selection

As will be set forth in greater detail below, inverse agonists and agonists to the non-endogenous, constitutively activated GPCR can be identified by the methodologies of this invention. Such inverse agonists and agonists are ideal candidates as lead compounds in drug discovery programs for treating diseases related to this receptor. Because of the ability to directly identify inverse agonists to the GPCR, thereby allowing for the development of pharmaceutical compositions, a search for diseases and disorders associated with the GPCR is relevant. The expression location of a receptor in a specific tissue provides a scientist with the ability to assign a physiological function to the receptor. For example, scanning both diseased and normal tissue samples for the presence of the GPCR now becomes more than an academic exercise or one which might be pursued along the path of identifying an endogenous ligand to the specific GPCR. Tissue scans can be conducted across a broad range of healthy and diseased tissues. Such tissue scans provide a potential first step in associating a specific receptor with a disease and/or disorder. Furthermore, expression of a receptor in diseased organs can assist one in determining the magnitude of clinical relevance of the receptor.

The DNA sequence of the GPCR can be used to make a probe/primer. In some preferred embodiments the DNA sequence is used to make a probe for (a) dot-blot analysis against tissue-mRNA, and/or (b) RT-PCR identification of the expression of the receptor in tissue samples. The presence of a receptor in a tissue source, or a diseased tissue, or the presence of the receptor at elevated concentrations in diseased tissue compared to a normal tissue, can be used to correlate location to function and indicate the receptor's physiological role/function and create a treatment regimen, including but not limited to, a disease associated with that function/role. Receptors can also be localized to regions of organs by this technique. Based on the known or assumed roles/functions of the specific tissues to which the receptor is localized, the putative physiological function of the receptor can be deduced. For example and not limitation, proteins located/expressed in areas of the thalamus are associated with sensorimotor processing and arousal (see, Goodman & Gilman's, The Pharmacological Basis of Therapeutics, 9^{th} Edition, page 465 (1996)). Proteins expressed in the hippocampus or in Schwann cells are associated with learning and memory, and myelination of peripheral nerves, respectively (see, Kandel, E. et al., Essentials of Neural Science and Behavior pages 657, 680 and 28, respectively (1995)).

E. Screening of Candidate Compounds

1. Generic GPCR Screening Assay Techniques

When a G protein receptor becomes constitutively active, it binds to a G protein (e.g. G_{q}, Gₛ, Gᵢ, G_{z}, Gₒ) and stimulates the binding of GTP to the G protein. The G protein then acts as a GTPase and hydrolyzes the GTP to GDP, whereby the receptor, under normal conditions, becomes deactivated. However, constitutively activated receptors continue to exchange GDP to GTP. A non-hydrolyzable analog of GTP, [³⁵S]GTPγS, can be used to monitor enhanced binding to membranes which express constitutively activated receptors. It is reported that [³⁵S]GTPγS can be used to monitor G protein coupling to membranes in the absence and presence of ligand. An example of this monitoring, among other examples well-known and available to those in the art, was reported by Traynor and Nahorski in 1995. The use of this assay system is typically for initial screening of candidate compounds because the system is generically applicable to all G protein-coupled receptors regardless of the particular G protein that interacts with the intracellular domain of the receptor.

2. Specific GPCR Screening Assay Techniques

Once candidate compounds are identified using the “generic” G protein-coupled receptor assay (i.e., an assay to select compounds that are agonists or inverse agonists), further screening to confirm that the compounds have interacted at the receptor site is preferred. For example, a compound identified by the “generic” assay may not bind to the receptor, but may instead merely “uncouple” the G protein from the intracellular domain.

a. Gₛ, G_{z} and Gᵢ.

Gₛ stimulates the enzyme adenylyl cyclase. Gᵢ (and G_{z} and Gₒ), on the other hand, inhibits adenylyl cyclase. Adenylyl cyclase catalyzes the conversion of ATP to cAMP; thus, constitutively activated GPCRs that couple the Gₛ protein are associated with increased cellular levels of cAMP. On the other hand, constitutively activated GPCRs that couple Gᵢ (or G_{z}, Gₒ) protein are associated with decreased cellular levels of cAMP. See, generally, “Indirect Mechanisms of Synaptic Transmission,” Chpt. 8, From Neuron To Brain (3^{rd} Ed.) Nichols, J. G. et al eds. Sinauer Associates, Inc. (1992). Thus, assays that detect cAMP can be utilized to determine if a candidate compound is, e.g., an inverse agonist to the receptor (i.e., such a compound would decrease the levels of cAMP). A variety of approaches known in the art for measuring cAMP can be utilized; a most preferred approach relies upon the use of anti-cAMP antibodies in an ELISA-based format. Another type of assay that can be utilized is a whole cell second messenger reporter system assay. Promoters on genes drive the expression of the proteins that a particular gene encodes. Cyclic AMP drives gene expression by promoting the binding of a cAMP-responsive DNA binding protein or transcription factor (CREB) that then binds to the promoter at specific sites (cAMP response elements) and drives the expression of the gene. Reporter systems can be constructed which have a promoter containing multiple cAMP response elements before the reporter gene, e.g., β-galactosidase or luciferase. Thus, a constitutively activated Gₛ-linked receptor causes the accumulation of cAMP that then activates the gene and leads to the expression of the reporter protein. The reporter protein such as β-galactosidase or luciferase can then be detected using standard biochemical assays (Chen et al. 1995).

b. Gₒ and G_{q}.

G_{q} and Gₒ are associated with activation of the enzyme phospholipase C, which in turn hydrolyzes the phospholipid PIP₂, releasing two intracellular messengers: diacycloglycerol (DAG) and inositol 1,4,5-triphoisphate (IP₃). Increased accumulation of IP₃ is associated with activation of G_{q}- and Gₒ-associated receptors. See, generally, “Indirect Mechanisms of Synaptic Transmission,” Chpt. 8, From Neuron To Brain (3^{rd} Ed.) Nichols, J. G. et al eds. Sinauer Associates, Inc. (1992). Assays that detect IP₃ accumulation can be utilized to determine if a candidate compound is, e.g., an inverse agonist to a G_{q}- or Gₒ-associated receptor (i.e., such a compound would decrease the levels of IP₃). G_{q}-associated receptors can also be examined using an AP1 reporter assay wherein G_{q}-dependent phospholipase C causes activation of genes containing AP1 elements; thus, activated G_{q}-associated receptors will evidence an increase in the expression of such genes, whereby inverse agonists thereto will evidence a decrease in such expression, and agonists will evidence an increase in such expression. Commercially available assays for such detection are available.

3. GPCR Fusion Protein

The use of an endogenous, constitutively activated GPCR or a non-endogenous, constitutively activated GPCR, for use in screening of candidate compounds for the direct identification of inverse agonists, agonists provide an interesting screening challenge in that, by definition, the receptor is active even in the absence of an endogenous ligand bound thereto. Thus, in order to differentiate between, e.g., the non-endogenous receptor in the presence of a candidate compound and the non-endogenous receptor in the absence of that compound, with an aim of such a differentiation to allow for an understanding as to whether such compound may be an inverse agonist or agonist or have no affect on such a receptor, it is preferred that an approach be utilized that can enhance such differentiation. A preferred approach is the use of a GPCR Fusion Protein.

Generally, once it is determined that a non-endogenous GPCR has been constitutively activated using the assay techniques set forth above (as well as others), it is possible to determine the predominant G protein that couples with the endogenous GPCR. Coupling of the G protein to the GPCR provides a signaling pathway that can be assessed. In some embodiments it is preferred that screening take place using a mammalian expression system, such a system will be expected to have endogenous G protein therein. Thus, by definition, in such a system, the non-endogenous, constitutively activated GPCR will continuously signal. In some embodiments it is preferred that this signal be enhanced such that in the presence of, e.g., an inverse agonist to the receptor, it is more likely that it will be able to more readily differentiate, particularly in the context of screening, between the receptor when it is contacted with the inverse agonist.

The GPCR Fusion Protein is intended to enhance the efficacy of G protein coupling with the non-endogenous GPCR. The GPCR Fusion Protein is preferred for screening with either an endogenous, constitutively active GPCR or a non-endogenous, constitutively activated GPCR because such an approach increases the signal that is utilized in such screening techniques. This is important in facilitating a significant “signal to noise” ratio; such a significant ratio is preferred for the screening of candidate compounds as disclosed herein.

The construction of a construct useful for expression of a GPCR Fusion Protein is within the purview of those having ordinary skill in the art. Commercially available expression vectors and systems offer a variety of approaches that can fit the particular needs of an investigator. Important criteria on the construction of such a GPCR Fusion Protein construct include but are not limited to, that the endogenous GPCR sequence and the G protein sequence both be in-frame (preferably, the sequence for the endogenous GPCR is upstream of the G protein sequence), and that the “stop” codon of the GPCR be deleted or replaced such that upon expression of the GPCR, the G protein can also be expressed. Other embodiments include constructs wherein the endogenous GPCR sequence and the G protein sequence are not in-frame and/or the “stop” codon is not deleted or replaced. The GPCR can be linked directly to the G protein, or there can be spacer residues between the two (preferably, no more than about 12, although this number can be readily ascertained by one of ordinary skill in the art). Based upon convenience it is preferred to use a spacer. Preferably, the G protein that couples to the non-endogenous GPCR will have been identified prior to the creation of the GPCR Fusion Protein construct. Because there are only a few G proteins that have been identified, it is preferred that a construct comprising the sequence of the G protein (i.e., a universal G protein construct (see Examples)) be available for insertion of an endogenous GPCR sequence therein; this provides for further efficiency in the context of large-scale screening of a variety of different endogenous GPCRs having different sequences.

As noted above, constitutively activated GPCRs that couple to Gᵢ, G_{z} and Gₒ are expected to inhibit the formation of cAMP making assays based upon these types of GPCRs challenging (i.e., the cAMP signal decreases upon activation thus making the direct identification of, e.g., inverse agonists (which would further decrease this signal), challenging. As will be disclosed herein, we have ascertained that for these types of receptors, it is possible to create a GPCR Fusion Protein that is not based upon the GPCRs endogenous G protein, in an effort to establish a viable cyclase-based assay. Thus, for example, an endogenous Gᵢ coupled receptor can be fused to a Gₛ protein—such a fusion construct, upon expression, “drives” or “forces” the endogenous GPCR to couple with, e.g., Gₛ rather than the “natural” Gᵢ protein, such that a cyclase-based assay can be established. Thus, for Gᵢ, G_{z} and Gₒ coupled receptors, in some embodiments it is preferred that when a GPCR Fusion Protein is used and the assay is based upon detection of adenylyl cyclase activity, that the fusion construct be established with Gₛ (or an equivalent G protein that stimulates the formation of the enzyme adenylyl cyclase).

| | | | Effect of | |
|---|---|---|---|---|
| | Effect of cAMP | Effect of IP₃ | cAMP | Effect on IP₃ |
| | Production upon | Accumulation | Production | Accumulat- |
| | Activation of | upon Activation | upon | ion upon |
| | GPCR (i.e., | of GPCR (i.e., | contact | contact |
| | constitutive | constitutive | with an | with an |
| G | activation or | activation or | Inverse | Inverse |
| protein | agonist binding) | agonist binding) | Agonist | Agonist |
| Gₛ | Increase | N/A | Decrease | N/A |
| Gᵢ | Decrease | N/A | Increase | N/A |
| G_{z} | Decrease | N/A | Increase | N/A |
| Gₒ | Decrease | Increase | Increase | Decrease |
| G_{q} | N/A | Increase | N/A | Decrease |

Equally effective is a G Protein Fusion construct that utilizes a G_{q} Protein fused with a Gₛ, Gᵢ, G_{z} or Gₒ Protein. In some embodiments a preferred fusion construct can be accomplished with a G_{q} Protein wherein the first six (6) amino acids of the G-protein α-subunit (“Gαq”) is deleted and the last five (5) amino acids at the C-terminal end of Gαq is replaced with the corresponding amino acids of the Gα of the G protein of interest. For example, a fusion construct can have a G_{q} (6 amino acid deletion) fused with a Gᵢ Protein, resulting in a “G_{q}/Gᵢ Fusion Construct”. This fusion construct will forces the endogenous Gᵢ coupled receptor to couple to its non-endogenous G protein, G_{q}, such that the second messenger, for example, inositol triphosphate or diacylgycerol, can be measured in lieu of cAMP production.

4. Co-transfection of a Target Gᵢ Coupled GPCR with a Signal-Enhancer Gₛ Coupled GPCR (cAMP Based Assays)

A Gᵢ coupled receptor is known to inhibit adenylyl cyclase, and, therefore, decreases the level of cAMP production, which can make assessment of cAMP levels challenging. An effective technique in measuring the decrease in production of cAMP as an indication of constitutive activation of a receptor that predominantly couples Gᵢ upon activation can be accomplished by co-transfecting a signal enhancer, e.g., a non-endogenous, constitutively activated receptor that predominantly couples with Gₛ upon activation (e.g., TSHR-A623I, disclosed below), with the Gᵢ linked GPCR. As is apparent, constitutive activation of a Gₛ coupled receptor can be determined based upon an increase in production of cAMP. Constitutive activation of a Gᵢ coupled receptor leads to a decrease in production cAMP. Thus, the co-transfection approach is intended to advantageously exploit these “opposite” affects. For example, co-transfection of a non-endogenous, constitutively activated Gₛ coupled receptor (the “signal enhancer”) with the endogenous Gᵢ coupled receptor (the “target receptor”) provides a baseline cAMP signal (i.e., although the Gᵢ coupled receptor will decrease cAMP levels, this “decrease” will be relative to the substantial increase in cAMP levels established by constitutively activated Gₛ coupled signal enhancer). By then co-transfecting the signal enhancer with a constitutively activated version of the target receptor, cAMP would be expected to further decrease (relative to base line) due to the increased functional activity of the Gᵢ target (i.e., which decreases cAMP).

Screening of candidate compounds using a cAMP based assay can then be accomplished, with two ‘changes’ relative to the use of the endogenous receptor/G-protein fusion: first, relative to the Gᵢ coupled target receptor, “opposite” effects will result, i.e., an inverse agonist of the Gᵢ coupled target receptor will increase the measured cAMP signal, while an agonist of the Gᵢ coupled target receptor will decrease this signal; second, as would be apparent, candidate compounds that are directly identified using this approach should be assessed independently to ensure that these do not target the signal enhancing receptor (this can be done prior to or after screening against the co-transfected receptors).

F. Medicinal Chemistry

Generally, but not always, direct identification of candidate compounds is conducted in conjunction with compounds generated via combinatorial chemistry techniques, whereby thousands of compounds are randomly prepared for such analysis. Generally, the results of such screening will be compounds having unique core structures; thereafter, these compounds may be subjected to additional chemical modification around a preferred core structure(s) to further enhance the medicinal properties thereof. Such techniques are known to those in the art and will not be addressed in detail in this patent document.

G. Pharmaceutical Compositions

Candidate compounds selected for further development can be formulated into pharmaceutical compositions using techniques well known to those in the art. Suitable pharmaceutically-acceptable carriers are available to those in the art; for example, see Remington's Pharmaceutical Sciences, 16^{th} Edition, 1980, Mack Publishing Co., (Osol et al., eds.).

H. Other Utilities

Although a preferred use of the non-endogenous versions of the GPCRs disclosed herein may be for the direct identification of candidate compounds as inverse agonists or agonists (preferably for use as pharmaceutical agents), other uses of these versions of GPCRs exist. For example, in vitro and in vivo systems incorporating GPCRs can be utilized to further elucidate and understand the roles these receptors play in the human condition, both normal and diseased, as well as understanding the role of constitutive activation as it applies to understanding the signaling cascade. In some embodiments it is preferred that the endogenous receptors be “orphan receptors”, i.e., the endogenous ligand for the receptor has not been identified. In some embodiments, therefore, the modified, non-endogenous GPCRs can be used to understand the role of endogenous receptors in the human body before the endogenous ligand therefore is identified. Such receptors can also be used to further elucidate known receptors and the pathways through which they transduce a signal. Other uses of the disclosed receptors will become apparent to those in the art based upon, inter alia, a review of this patent document.

### EXAMPLES

The following examples are presented for purposes of elucidation, and not limitation, of the present invention. While specific nucleic acid and amino acid sequences are disclosed herein, those of ordinary skill in the art are credited with the ability to make minor modifications to these sequences while achieving the same or substantially similar results reported below. The traditional approach to application or understanding of sequence cassettes from one sequence to another (e.g. from rat receptor to human receptor or from human receptor A to human receptor B) is generally predicated upon sequence alignment techniques whereby the sequences are aligned in an effort to determine areas of commonality. The mutational approach disclosed herein does not rely upon this approach but is instead based upon an algorithmic approach and a positional distance from a conserved proline residue located within the TM6 region of human GPCRs. Once this approach is secured, those in the art are credited with the ability to make minor modifications thereto to achieve substantially the same results (i.e., constitutive activation) disclosed herein. Such modified approaches are considered within the purview of this disclosure.

### Example 1

Endogenous Human GPCRs

1. Identification of Human GPCRs

The disclosed endogenous human GPCRs were identified based upon a review of the GenBank™ database information. While searching the database, the following cDNA clones were identified as evidenced below (Table C).

TABLE C

| Disclosed | | Open | | | |
|---|---|---|---|---|---|
| Human | Accession | Reading | Reference To | | |
| Orphan | Number | Frame | Homologous | Nucleic Acid | Amino Acid |
| GPCRs | Identified | (Base Pairs) | GPCR | SEQ. ID. NO. | SEQ. ID. NO. |
| hRUP28 | AC073957 | 1,002 bp | hGPR30 | 1 | 2 |
| hRUP29 | AC083865 | 918 bp | hGPR18 | 3 | 4 |
| hRUP30 | AC055863 | 1,125 bp | hBRB1 | 5 | 6 |
| hRUP31 | AL356214 | 1,086 bp | hGALR-1 | 7 | 8 |
| hRUP32 | AL513524 | 1,038 bp | hPNR | 9 | 10 |
| hRUP33 | AL513524 | 1,020 bp | GPR57 | 11 | 12 |
| | | | GPR58 | | |
| hRUP34 | AL513524 | 1,029 bp | hPNR | 13 | 14 |
| hRUP35 | AC021089 | 1,062 bp | hκ-type 3 | 15 | 16 |
| | | | opioid | | |
| hRUP36 | AC090099 | 969 bp | GPR90 | 17 | 18 |
| hRUP37 | AC090099 | 969 bp | hMRG | 19 | 20 |

2. Full Length Cloning

a. hRUP28 (Seq. Id. Nos. 1 & 2)

The disclosed human RUP28 was identified based upon the use of GenBank database information. While searching the database, a cDNA clone with Accession Number AC073957 was identified as a human genomic sequence from chromosome 7.

The full length RUP28 was cloned by PCR using primers: 5′-CAGAGCTCTGGTGGCCACCTCTGTCC-3′ (SEQ. ID. NO.:21; sense, 5′ of initiation codon), 5′-CTGCGTCCACCAGAGTCACGTCTCC-3′ (SEQ. ID. NO.:22; antisense, 3′ of stop codon), and human adult liver Marathon-Ready™ cDNA (Clontech) as template. Advantage™ cDNA polymerase (Clontech) was used for the amplification in a 50 μl reaction by the following cycle with step 2 to 4 repeated 35 times: 95° C. for 5 min; 94° C. for 30 sec; 58° C. for 30 sec; 72° C. for 1 min 30 sec; and 72° C. for 7 min.

A 1.16 kb PCR fragment was isolated from a 1% agarose gel and cloned into the pCRII-TOPO vector (Invitrogen) and sequenced using the ABI Big Dye Terminator Kit (P.E. Biosystems). See, SEQ. ID. NO.:1 for the nucleic acid sequence and SEQ. ID. NO.:2 for the putative amino acid sequence.

b. hRUP29 (Seq. Id. Nos. 3 & 4)

The disclosed human RUP29 was identified based upon the use of GenBank database information. While searching the database, a cDNA clone with Accession Number AC0083865 was identified as a human genomic sequence from chromosome 7.

The full length RUP29 was cloned by PCR using primers: 5′-GTATGCCTGGCCACAATACCTCCAGG-3′ (SEQ. ID. NO.:23; sense, containing the initiation codon), 5′-GTTTGTGGCTAACGGCACAAAACACAATTCC-3′ (SEQ. ID. NO.:24; antisense, containing the stop codon) and human genomic DNA as template. TaqPlus® Precision DNA polymerase (Stratagene) was used for the amplification in a 50 μl reaction by the following cycle with step 2 to 4 repeated 35 times: 94° C. for 5 min; 94° C. for 30 sec; 54° C. for 30 sec; 72° C. for 1 min 30 sec; and 72° C. for 7 min.

A 930 bp PCR fragment was isolated from a 1% agarose gel and cloned into the pCRII-TOPO vector (Invitrogen) and sequenced using the ABI Big Dye Terminator Kit (P.E. Biosystems).

Rapid amplification of cDNA ends (RACE) was performed using human leukocyte and ovary Marathon-Ready™ cDNA (Clontech) to determine the precise 5′ end of RUP29 cDNA. RUP29 specific primer (1) having the sequence: 5-GGTACCACAATGACAATCACCAGCGTCC-3′(SEQ. ID. NO.:25) and AP1 primer (Clontech) were used for the first-round PCR reaction, and RUP29 specific primer (2) having the following sequence: 5′-GGAACGTGAGGTACATGTGGATGTGCAGC-3′ (SEQ. ID. NO.:26) and AP2 primer (Clontech) were used for the second-round PCR reaction. The products of the RACE reactions were isolated and cloned into the pCRII-TOPO vector (Invitrogen) and sequenced. See, SEQ. ID. NO.:3 for the nucleic acid sequence and SEQ. ID. NO.:4 for the putative amino acid sequence.

c. hRUP30 (Seq. Id. Nos. 5 & 6)

The disclosed human RUP30 was identified based upon the use of GenBank database information. While searching the database, a cDNA clone with Accession Number AC055863 was identified as a human genomic sequence from chromosome 17.

The full length RUP30 was cloned by 5′RACE-PCR with a human pancreas Marathon-Ready™ cDNA (Clontech) as template and the following oligonucleotide: 5′-GCAGTGTAGCGGTCAACCGTGAGCAGG-3′(SEQ. ID. NO.:27; sense, containing the initiation codon), and AP1 primer (Clontech) were used for the first round of RT-PCR and oligonucleotide: 5′-TGAGCAGGATGGCGATCCAGACTGAGGCGTGG-3′(SEQ. ID. NO.:28; antisense, containing the stop codon) and AP2 primer (Clontech) were used for the second round of PCR. DNA fragments generated by the 5′ RACE-PCR were cloned into the pCRII-TOPO vector (Invitrogen) and sequenced using the SP6/T7 primers (Stratagene).

Based on the sequence of the 5′ RACE products, the full length RUP30 was cloned by RT-PCR, using primers: 5′-GAGGTACAGCTGGCGATGCTGACAG-3′ (SEQ. ID. NO.:29; sense, ATG as the initiation codon); 5′-GTGGCCATGAGCCACCCTGAGCTCC-3′ (SEQ. ID. NO.:30; antisense, 3′ of the stop codon) and human pancreas Marathon-Ready™ cDNA (Clontech) as template. Taq DNA polymerase (Stratagene) was used for the amplification in 50 μl reaction by the following cycle with step 2 to step 4 repeated 35 times: 94° C. for 40 seconds; 94° C. for 20 seconds; 64° C. for 20 seconds; 72° C. for 2 minutes; and 72° C. for 5 minutes.

A 1.2 Kb PCR fragment was isolated from a 1% agarose gel and cloned into the pCRII-TOPO vector (Invitrogen) and several clones were sequenced using the ABI Big Dye Terminator kit (P.E. Biosystems). See, SEQ. ID. NO.:5 for the nucleic acid sequence and SEQ. ID. NO.:6 for the putative amino acid sequence.

d. hRUP31 (Seq. Id. Nos. 7 & 8)

The disclosed human RUP31 was identified based upon the use of GenBank database information. While searching the database, a cDNA clone with Accession Number AL356214 was identified as a human genomic sequence from chromosome 10.

The full length RUP31 was cloned by RT-PCR using primers: 5′-GGAATGTCCACTGAATGCGCGCGG-3′ (SEQ. ID. NO.:31; sense, containing the initiation codon), 5′-AGCTCGCCAGGTGTGAGAAACTCGG-3′ (SEQ. ID. NO.:32; antisense, 3′ of stop codon) and human mammary gland Marathon-Ready™ cDNA (Clontech) as template. Advantage™ cDNA polymerase (Clontech) was used for the amplification in 50 μl reaction by the following cycle with step 2 to step 4 repeated 35 times: 94° C. for 40 sec; 94° C. for 20 sec; 66° C. for 20 sec; 72° C. for 1 min 30 sec; and 72° C. for 5 min.

A 1.1 kb PCR fragment was isolated from a 1% agarose gel and cloned into the pCRII-TOPO vector (Invitrogen) and sequenced using the ABI Big Dye Terminator Kit (P.E. Biosystems). See, SEQ. ID. NO.:7 for the nucleic acid sequence and SEQ. ID. NO.:8 for the putative amino acid sequence.

e. hRUP32 (Seq. Id. Nos. 9 & 10)

The disclosed human RUP32 was identified based upon the use of GenBank database information. While searching the database, a cDNA clone with Accession Number AL513524 was identified as a human genomic sequence from chromosome 6.

The full length RUP32 was cloned by PCR using primers: 5′-GCGTTATGAGCAGCAATTCATCCCTGCTGG-3′ (SEQ. ID. NO.:33; sense, containing the initiation codon), 5′-GTATCCTGAACTTCGTCTATACAACTGC-3′ (SEQ. ID. NO.:34; antisense) and human genomic DNA (Clontech) as template. TaqPlus® Precision DNA polymerase (Stratagene) was used for the amplification by the following cycle with step 2 to step 4 repeated 35 times: 94° C. for 3 min; 94° C. for 20 sec; 58° C. for 20 sec; 72° C. for 1 min 30 sec; and 72° C. for 7 min.

A 1.06 kb PCR fragment was isolated from a 1% agarose gel and cloned into the pCRII-TOPO vector (Invitrogen) and sequenced using the ABI Big Dye Terminator Kit (P.E. Biosystems). See, SEQ. ID. NO.:9 for the nucleic acid sequence and SEQ. ID. NO.: 10 for the putative amino acid sequence.

f. hRUP33 (Seq. Id. Nos. 11 & 12)

The disclosed human RUP33 was identified based upon the use of GenBank database information. While searching the database, a cDNA clone with Accession Number AL513524 was identified as a human genomic sequence from chromosome 6.

The full length RUP33 was cloned by PCR using primers: 5′-CCCTCAGGAATGATGCCCTTTTGCCACAA-3′ (SEQ. ID. NO.:35; sense, containing the initiation codon), 5′-ATCCATGTGGTTGGTGCATGTGGTTCGT-3′ (SEQ. ID. NO.:36; antisense) and human genomic DNA (Clontech) as template. TaqPlus® Precision DNA polymerase (Stratagene) was used for the amplification by the following cycle with step 2 to step 4 repeated 35 times: 94° C. for 3 min; 94° C. for 20 sec; 56° C. for 20 sec; 72° C. for 1 min 30 sec; and 72° C. for 7 min.

A 1.1 kb PCR fragment was isolated from a 1% agarose gel and cloned into the pCRII-TOPO vector (Invitrogen) and sequenced using the ABI Big Dye Terminator Kit (P.E. Biosystems). See, SEQ. ID. NO.:11 for the nucleic acid sequence and SEQ. ID. NO.: 12 for the putative amino acid sequence.

g. hRUP34 (Seq. Id. Nos. 13 & 14)

The disclosed human RUP34 was identified based upon the use of GenBank database information. While searching the database, a cDNA clone with Accession Number AL513524 was identified as a human genomic sequence from chromosome 6.

The full length RUP34 was cloned by PCR using primers: 5′-AAACAACAAACAGCAGAACCATGACCAGC-3′ (SEQ. ID. NO.:37; sense, containing the initiation codon), 5′-ACATAGAGACAAGTGACATGTGTGAACCAC-3′ (SEQ. ID. NO.:38; antisense) and human genomic DNA (Clontech) as template. TaqPlus® Precision DNA polymerase (Stratagene) was used for the amplification by the following cycle with step 2 to step 4 repeated 35 times: 94° C. for 3 min; 94° C. for 20 sec; 60° C. for 20 sec; 72° C. for 1 min 30 sec; and 72° C. for 7 min.

A 1.27 kb PCR fragment was isolated from a 1% agarose gel and cloned into the pCRII-TOPO vector (Invitrogen) and sequenced using the ABI Big Dye Terminator Kit (P.E. Biosystems). See, SEQ. ID. NO.: 13 for the nucleic acid sequence and SEQ. ID. NO.: 14 for the putative amino acid sequence.

h. hRUP35 (Seq. Id. Nos. 15 & 16)

The disclosed human RUP35 was identified based upon the use of GenBank database information. While searching the database, a cDNA clone with Accession Number AC021089 was identified as a human genomic sequence from chromosome 16.

The 5′ sequence of RUP35 was determined by 5′ RACE-PCR with a human fetal brain Marathon-Ready™ cDNA (Clontech) as template. Oligonucleotide 5′-GGTATGAGACCGTGTGGTACTTGAGC-3′ (SEQ. ID. NO.:39; sense) and AP1 primer (Clontech) were used for the first round of RT-PCR and oligonucleotide 5′-GTGGCAGACAGCGATATACCTGTCAATGG-3′ (SEQ. ID. NO.:40; antisense) and AP2 primer (Clontech) were used for the second round of PCR. DNA fragments generated by the 5′ RACE-PCR were cloned into the pCRII-TOPO vector (Invitrogen) and sequenced using the SP6/T7 primers (Stratagene).

Based upon the sequence of the 5′ RACE products, the full length RUP35 was cloned by RT-PCR, using primers 5′-GCGCTCATGGAGCACACGCACGCCCAC-3′ (SEQ. ID. NO.:41; sense, ATG as the initiation codon) and 5′-GAGGCAGTAGTTGCCACACCTATGG-3′ (SEQ. ID. NO.:42; antisense, 3′ of the stop codon) and human brain Marathon-Ready™ cDNA (Clontech) as template. Advantage™ cDNA polymerase (Clontech) was used for the amplification in 100 μl reaction by the following cycle with step 2 to step 4 repeated 45 times: 95° C. for 2 min; 95° C. for 20 sec; 60° C. for 20 sec; 72° C. for 1 min 30 sec; and 72° C. for 5 min.

A 1.0 kb PCR fragment was isolated from a 1% agarose gel and cloned into the pCRII-TOPO vector (Invitrogen) and sequenced using the ABI Big Dye Terminator Kit (P.E. Biosystems). See, SEQ. ID. NO.:15 for the nucleic acid sequence and SEQ. ID. NO.:16 for the putative amino acid sequence.

i. hRUP36 (Seq. Id. Nos. 17 & 18)

The disclosed human RUP36 was identified based upon the use of GenBank database information. While searching the database, a cDNA clone with Accession Number AC090099 was identified as a human genomic sequence from chromosome 11.

The full length RUP36 was cloned by PCR using primers: 5′-CATCTGGTTTGTGTTCCCAGGGGCACCAG-3′ (SEQ. ID. NO.:43; sense, 5′ of start codon), 5′-GACAGTGTTGCTCTCAAAGTCCCGTCTGACTG-3′ (SEQ. ID. NO.:44; antisense, 3′ of stop codon) and human genomic DNA (Clontech) as template. TaqPlus® Precision DNA polymerase (Stratagene) was used for the amplification in a 50 μl reaction by the following cycle with step 2 to step 4 repeated 30 times: 95° C., 5 min; 95° C. for 30 sec; 70° C. for 30 sec; 72° C. for 1 min 30 sec; and 72° C. for 7 min.

A 1.0 kb PCR fragment was isolated from a 1% agarose gel and cloned into the pCRII-TOPO vector (Invitrogen) and sequenced using the ABI Big Dye Terminator Kit (P.E. Biosystems). See, SEQ. ID. NO.:17 for the nucleic acid sequence and SEQ. ID. NO.: 18 for the putative amino acid sequence.

j. hRUP37 (Seq. Id. Nos. 19 & 20)

The disclosed human RUP37 was identified based upon the use of GenBank database information. While searching the database, a cDNA clone with Accession Number AC090099 was identified as a human genomic sequence from chromosome 11.

The full length RUP37 was cloned by PCR using primers: 5′-CTGTTTCCAGGGTCATCAGACTGGG-3′ (SEQ. ID. NO.:45; sense); 5′-GCAGCATTGCTCTCAAAGTCCTGTCTG-3′ (SEQ. ID. NO.:46; antisense) and human genomic DNA (Clontech) as template. TaqPlus® Precision DNA polymerase (Stratagene) was used for the amplification by the following cycle with step 2 to step 4 repeated 35 times: 95° C. for 5 min; 95° C. for 30 sec; 62° C. for 30 sec; 72° C. for 1 min 30 sec; and 72° C. for 7 min.

A 969 base pair was isolated from a 1%% agarose gel and cloned into the pCRII-TOPO vector (Invitrogen) and sequenced using the ABI Big Dye Terminator Kit (P.E. Biosystems). See, SEQ. ID. NO.:19 for the nucleic acid sequence and SEQ. ID. NO.:20 for the putative amino acid sequence.

### Example 2

Preparation of Non-Endogenous, Constitutively Activated GPCRs

Those skilled in the art are credited with the ability to select techniques for mutation of a nucleic acid sequence. Presented below are approaches utilized to create non-endogenous versions of several of the human GPCRs disclosed above. The mutations disclosed below are based upon an algorithmic approach whereby the 16^{th} amino acid (located in the IC3 region of the GPCR) from a conserved proline (or an endogenous, conservative substitution therefore) residue (located in the TM6 region of the GPCR, near the TM6/IC3 interface) is mutated, preferably to an alanine, histamine, arginine or lysine amino acid residue, most preferably to a lysine amino acid residue.

1. Transformer Site-Directed™ Mutagenesis

Preparation of non-endogenous human GPCRs may be accomplished on human GPCRs using, inter alia, Transformer Site-Directed™ Mutagenesis Kit (Clontech) according to the manufacturer instructions. In some embodiments two mutagenesis primers are used, preferably a lysine mutagenesis oligonucleotide that creates the lysine mutation, and a selection marker oligonucleotide. For convenience, the codon mutation to be incorporated into the human GPCR is also noted, in standard form (Table D):

TABLE D

| Receptor Identifier | Codon Mutation |
|---|---|
| hRUP28 | V274K |
| hRUP29 | T249K |
| hRUP30 | R232K |
| hRUP31 | M294K |
| hRUP32 | F220K |
| hRUP34 | A238K |
| hRUP35 | Y215K |
| hRUP36 | L294K |
| hRUP37 | T219K |

### Example 3

Receptor Expression

Although a variety of cells are available to the art-skilled for the expression of proteins, it is preferred that mammalian cells be utilized. The primary reason for this is predicated upon practicalities, i.e., utilization of, e.g., yeast cells for the expression of a GPCR, while possible, introduces into the protocol a non-mammalian cell which may not (indeed, in the case of yeast, does not) include the receptor-coupling, genetic-mechanism and secretary pathways that have evolved for mammalian systems—thus, results obtained in non-mammalian cells, while of potential use, are not as preferred as those obtained using mammalian cells. Of the mammalian cells, COS-7, 293 and 293T cells are particularly preferred, although the specific mammalian cell utilized can be predicated upon the particular needs of the artisan.

a. Transient Transfection

On day one, 6×10⁶ cells/10 cm dish of 293 cells well were plated out. On day two, two reaction tubes were prepared (the proportions to follow for each tube are per plate): tube A was prepared by mixing 4 μg DNA (e.g., pCMV vector; pCMV vector with receptor cDNA, etc.) in 0.5 ml serum free DMEM (Gibco BRL); tube B was prepared by mixing 24 μl lipofectamine (Gibco BRL) in 0.5 ml serum free DMEM. Tubes A and B were admixed by inversion (several times), followed by incubation at room temperature for 30-45 min. The admixture is referred to as the “transfection mixture”. Plated 293 cells were washed with 1×PBS, followed by addition of 5 ml serum free DMEM. One ml of the transfection mixture were added to the cells, followed by incubation for 4 hrs at 37° C./5% CO₂. The transfection mixture was removed by aspiration, followed by the addition of 10 ml of DMEM/10% Fetal Bovine Serum. Cells were incubated at 37° C./5% CO₂. After 48 hr incubation, cells were harvested and utilized for analysis.

b. Stable Cell Lines

Approximately 12×10⁶ 293 cells will be plated on a 15 cm tissue culture plate, and grown in DME High Glucose Medium containing 10% fetal bovine serum and one percent sodium pyruvate, L-glutamine, and antibiotics. Twenty-four hours following plating of 293 cells (to approximately ˜80% confluency), the cells will be transfected using 12 μg of DNA. The 12 μg of DNA is combined with 60 μl of lipofectamine and 2 mL of DME High Glucose Medium without serum. The medium will be aspirated from the plates and the cells washed once with medium without serum. The DNA, lipofectamine, and medium mixture will be added to the plate along with 10 mL of medium without serum. Following incubation at 37° C. for four to five hours, the medium will be aspirated and 25 ml of medium containing serum will be added. Twenty-four hours following transfection, the medium will be aspirated again, and fresh medium with serum will be added. Forty-eight hours following transfection, the medium will be aspirated and medium with serum will be added containing geneticin (G418 drug) at a final concentration of 500 μg/mL. The transfected cells will then undergo selection for positively transfected cells containing the G418 resistant gene. The medium will be replaced every four to five days as selection occurs. During selection, cells will be grown to create stable pools, or split for stable clonal selection.

### Example 4

Assays for Determination of Constitutive Activity of Non-Endogenous GPCRs

A variety of approaches are available for assessment of constitutive activity of the non-endogenous human GPCRs. The following are illustrative; those of ordinary skill in the art are credited with the ability to determine those techniques that are preferentially beneficial for the needs of the artisan.

1. Membrane Binding Assays: [³⁵S]GTPγS Assay

When a G protein-coupled receptor is in its active state, either as a result of ligand binding or constitutive activation, the receptor couples to a G protein and stimulates the release of GDP and subsequent binding of GTP to the G protein. The alpha subunit of the G protein-receptor complex acts as a GTPase and slowly hydrolyzes the GTP to GDP, at which point the receptor normally is deactivated. Constitutively activated receptors continue to exchange GDP for GTP. The non-hydrolyzable GTP analog, [³⁵S]GTPγS, can be utilized to demonstrate enhanced binding of [³⁵S]GTPγS to membranes expressing constitutively activated receptors. Advantages of using [³⁵S]GTPγS binding to measure constitutive activation include but are not limited to the following: (a) it is generically applicable to all G protein-coupled receptors; (b) it is proximal at the membrane surface making it less likely to pick-up molecules which affect the intracellular cascade.

The assay takes advantage of the ability of G protein coupled receptors to stimulate [³⁵S]GTPγS binding to membranes expressing the relevant receptors. The assay can, therefore, be used in the direct identification method to screen candidate compounds to constitutively activated G protein-coupled receptors. The assay is generic and has application to drug discovery at all G protein-coupled receptors.

The [³⁵S]GTPγS assay is incubated in 20 mM HEPES and between 1 and about 20 mM MgCl₂ (this amount can be adjusted for optimization of results, although 20 mM is preferred) pH 7.4, binding buffer with between about 0.3 and about 1.2 nM [³⁵S]GTPγS (this amount can be adjusted for optimization of results, although 1.2 is preferred) and 12.5 to 75 μg membrane protein (e.g., 293 cells expressing the Gₛ Fusion Protein; this amount can be adjusted for optimization) and 10 μM GDP (this amount can be changed for optimization) for 1 hour. Wheatgerm agglutinin beads (25 μl; Amersham) will then be added and the mixture incubated for another 30 minutes at room temperature. The tubes will be then centrifuged at 1500×g for 5 minutes at room temperature and then counted in a scintillation counter.

2. Adenylyl Cyclase

A Flash Plate™ Adenylyl Cyclase kit (New England Nuclear; Cat. No. SMP004A) designed for cell-based assays can be modified for use with crude plasma membranes. The Flash Plate wells can contain a scintillant coating which also contains a specific antibody recognizing cAMP. The cAMP generated in the wells can be quantitated by a direct competition for binding of radioactive cAMP tracer to the cAMP antibody. The following serves as a brief protocol for the measurement of changes in cAMP levels in whole cells that express the receptors.

Transfected cells will be harvested approximately twenty four hours after transient transfection. Media will be carefully aspirated and discarded. Ten ml of PBS will gently be added to each dish of cells followed by careful aspiration. One ml of Sigma cell dissociation buffer and 3 ml of PBS will be added to each plate. Cells will be pipetted off the plate and the cell suspension collected into a 50 ml conical centrifuge tube. Cells will be centrifuged at room temperature at 1,100 rpm for 5 min. The cell pellet will be carefully re-suspended into an appropriate volume of PBS (about 3 ml/plate). The cells will be then counted using a hemocytometer and additional PBS will be added to give the appropriate number of cells (to a final volume of about 50 μl/well). cAMP standards and Detection Buffer (comprising 1 μCi of tracer [¹²⁵I cAMP (50 μl] to 11 ml Detection Buffer) will be prepared and maintained in accordance with the manufacturer's instructions. Assay Buffer will be prepared fresh for screening and contained 50 μl of Stimulation Buffer, 3 μl of test compound (12 μM final assay concentration) and 50 μl cells, Assay Buffer will be stored on ice until utilized. The assay will be initiated by addition of 50 μl of cAMP standards to appropriate wells followed by addition of 50 μl of PBSA to wells H-11 and H12. Fifty μl of Stimulation Buffer will be added to all wells. DMSO (or selected candidate compounds) will be added to appropriate wells using a pin tool capable of dispensing 3 μl of compound solution, with a final assay concentration of 12 μM test compound and 100 μl total assay volume. The cells will then be added to the wells and incubated for 60 min at room temperature. One hundred μl of Detection Mix containing tracer cAMP will then be added to the wells. Plates will be incubated for an additional 2 hours followed by counting in a Wallac MicroBeta™ scintillation counter. Values of cAMP/well will then be extrapolated from a standard cAMP curve which will be contained within each assay plate.

3. Cell-Based cAMP for Gᵢ Coupled Target GPCRs

TSHR is a Gₛ coupled GPCR that causes the accumulation of cAMP upon activation. TSHR will be constitutively activated by mutating amino acid residue 623 (i.e., changing an alanine residue to an isoleucine residue). A Gᵢ coupled receptor is expected to inhibit adenylyl cyclase, and, therefore, decrease the level of cAMP production, which can make assessment of cAMP levels challenging. An effective technique for measuring the decrease in production of cAMP as an indication of constitutive activation of a Gᵢ coupled receptor can be accomplished by co-transfecting, most preferably, non-endogenous, constitutively activated TSHR (TSHR-A623I) (or an endogenous, constitutively active Gₛ coupled receptor) as a “signal enhancer” with a Gᵢ linked target GPCR to establish a baseline level of cAMP. Upon creating a non-endogenous version of the Gᵢ coupled receptor, this non-endogenous version of the target GPCR is then co-transfected with the signal enhancer, and it is this material that can be used for screening. This approach will be utilized to effectively generate a signal when a cAMP assay is used; this approach is preferably used in the direct identification of candidate compounds against Gᵢ coupled receptors. It is noted that for a Gᵢ coupled GPCR, when this approach is used, an inverse agonist of the target GPCR will increase the cAMP signal and an agonist will decrease the cAMP signal.

On day one, 2×10⁴ 293 cells/well will be plated out. On day two, two reaction tubes will be prepared (the proportions to follow for each tube are per plate): tube A will be prepared by mixing 2 ug DNA of each receptor transfected into the mammalian cells, for a total of 4 ug DNA (e.g., pCMV vector; pCMV vector with mutated THSR (TSHR-A623I); TSHR-A6231 and GPCR, etc.) in 1.2 ml serum free DMEM (Irvine Scientific, Irvine, Calif.); tube B will be prepared by mixing 120 μl lipofectamine (Gibco BRL) in 1.2 ml serum free DMEM. Tubes A and B will then be admixed by inversion (several times), followed by incubation at room temperature for 30-45 min. The admixture is referred to as the “transfection mixture”. Plated 293 cells will be washed with 1×PBS, followed by addition of 10 ml serum free DMEM. 2.4 ml of the transfection mixture will then be added to the cells, followed by incubation for 4 hrs at 37° C./5% CO₂. The transfection mixture will then be removed by aspiration, followed by the addition of 25 ml of DMEM/10% Fetal Bovine Serum. Cells will then be incubated at 37° C./5% CO₂. After 24 hr incubation, cells will be harvested and utilized for analysis.

A Flash Plate™ Adenylyl Cyclase kit (New England Nuclear; Cat. No. SMP004A) although designed for cell-based assays, can be modified for use with crude plasma membranes depending on the need of the skilled artisan. The Flash Plate wells will contain a scintillant coating which also contains a specific antibody recognizing cAMP. The cAMP generated in the wells can be quantified by a direct competition for binding of radioactive cAMP tracer to the cAMP antibody. The following serves as a brief protocol for the measurement of changes in cAMP levels in whole cells that express the receptors.

Transfected cells will be harvested approximately twenty four hours after transient transfection. Media will be carefully aspirated and discarded. Ten ml of PBS will be gently added to each dish of cells followed by careful aspiration. One ml of Sigma cell dissociation buffer and 3 ml of PBS will be added to each plate. Cells will be pipetted off the plate and the cell suspension will be collected into a 50 ml conical centrifuge tube. Cells will be centrifuged at room temperature at 1,100 rpm for 5 min. The cell pellet will be carefully re-suspended into an appropriate volume of PBS (about 3 ml/plate). The cells will then be counted using a hemocytometer and additional PBS is added to give the appropriate number of cells (to a final volume of about 50 μl/well). cAMP standards and Detection Buffer (comprising 1 μCi of tracer [¹²⁵I cAMP (50 μl] to 11 ml Detection Buffer) will be prepared and maintained in accordance with the manufacturer's instructions. Assay Buffer should be prepared fresh for screening and contained 50 μl of Stimulation Buffer, 311 of test compound (12 μM final assay concentration) and 5011 cells, Assay Buffer can be stored on ice until utilized. The assay can be initiated by addition of 50 μl of cAMP standards to appropriate wells followed by addition of 50 μl of PBSA to wells H-11 and H12. Fifty μl of Stimulation Buffer will be added to all wells. Selected compounds (e.g., TSH) will be added to appropriate wells using a pin tool capable of dispensing 3 μl of compound solution, with a final assay concentration of 12 μM test compound and 100 μl total assay volume. The cells will then be added to the wells and incubated for 60 min at room temperature. One hundred μl of Detection Mix containing tracer cAMP will then be added to the wells. Plates will then be incubated additional 2 hours followed by counting in a Wallac MicroBeta scintillation counter. Values of cAMP/well will then be extrapolated from a standard cAMP curve which is contained within each assay plate.

4. Reporter-Based Assays

a. CRE-Luc Reporter Assay (Gₛ-Associated Receptors)

293 and 293T cells will be plated-out on 96 well plates at a density of 2×10⁴ cells per well and will be transfected using Lipofectamine Reagent (BRL) the following day according to manufacturer instructions. A DNA/lipid mixture will be prepared for each 6-well transfection as follows: 260 ng of plasmid DNA in 100 μl of DMEM are gently mixed with 2 μl of lipid in 100 μl of DMEM (the 260 ng of plasmid DNA consisted of 200 ng of a 8×CRE-Luc reporter plasmid, 50 ng of pCMV comprising endogenous receptor or non-endogenous receptor or pCMV alone, and 10 ng of a GPRS expression plasmid (GPRS in pcDNA3 (Invitrogen)). The 8×CRE-Luc reporter plasmid is prepared as follows: vector SRIF-β-gal will be obtained by cloning the rat somatostatin promoter (−71/+51) at BglV-HindIII site in the pβgal-Basic Vector (Clontech). Eight (8) copies of cAMP response element will be obtained by PCR from an adenovirus template AdpCF126CCRE8 (see, 7 Human Gene Therapy 1883 (1996)) and cloned into the SRIF-β-gal vector at the Kpn-BglV site, resulting in the 8×CRE-β-gal reporter vector. The 8×CRE-Luc reporter plasmid will be generated by replacing the beta-galactosidase gene in the 8×CRE-β-gal reporter vector with the luciferase gene obtained from the pGL3-basic vector (Promega) at the HindIII-BamHI site. Following 30 min. incubation at room temperature, the DNA/lipid mixture will be diluted with 400 μl of DMEM and 100 μl of the diluted mixture will be added to each well. One hundred μl of DMEM with 10% FCS will be added to each well after a 4 hr incubation in a cell culture incubator. The following day the transfected cells will be changed with 200 μl/well of DMEM with 10% FCS. Eight hours later, the wells will be changed to 100 μl/well of DMEM without phenol red, after one wash with PBS. Luciferase activity will be measured the next day using the LucLite™ reporter gene assay kit (Packard) following manufacturer's instructions and read on a 1450 MicroBeta™ scintillation and luminescence counter (Wallac).

b. AP1 Reporter Assay (G_{q}-Associated Receptors)

A method to detect G_{q} stimulation depends on the known property of G_{q}-dependent phospholipase C to cause the activation of genes containing AP1 elements in their promoter. A Pathdetect™ AP-1 cis-Reporting System (Stratagene, Catalogue #219073) can be utilized following the protocol set forth above with respect to the CREB reporter assay, except that the components of the calcium phosphate precipitate were 410 ng pAPI-Luc, 80 ng pCMV-receptor expression plasmid, and 20 ng CMV-SEAP.

C. SRF-Luc Reporter Assay (G_{q}-Associated Receptors)

One method to detect G_{q} stimulation depends on the known property of G_{q}-dependent phospholipase C to cause the activation of genes containing serum response factors in their promoter. A Pathdetect™ SRF-Luc-Reporting System (Stratagene) can be utilized to assay for G_{q} coupled activity in, e.g., COS7 cells. Cells are transfected with the plasmid components of the system and the indicated expression plasmid encoding endogenous or non-endogenous GPCR using a Mammalian Transfection™ Kit (Stratagene, Catalogue #200285) according to the manufacturer's instructions. Briefly, 410 ng SRF-Luc, 80 ng pCMV-receptor expression plasmid and 20 ng CMV-SEAP (secreted alkaline phosphatase expression plasmid; alkaline phosphatase activity is measured in the media of transfected cells to control for variations in transfection efficiency between samples) are combined in a calcium phosphate precipitate as per the manufacturer's instructions. Half of the precipitate is equally distributed between 3 wells in a 96-well plate, kept on the cells in a serum free media for 24 hours. The last 5 hours the cells are incubated with 1 μM Angiotensin, where indicated. Cells are then lysed and assayed for luciferase activity using a Luclite™ Kit (Packard, Cat. #6016911) and “Trilux 1450 Microbeta” liquid scintillation and luminescence counter (Wallac) as per the manufacturer's instructions. The data can be analyzed using GraphPad PriSm™ 2.0a (GraphPad Software Inc.).

d. Intracellular IP₃ Accumulation Assay (G_{q}-Associated Receptors)

On day 1, cells comprising the receptors (endogenous and/or non-endogenous) are plated onto 24 well plates, usually 1×10⁵ cells/well (although his number can be optimized. On day 2 cells are transfected by firstly mixing 0.25 ug DNA in 50 μl serum free DMEM/well and 2 μl lipofectamine in 50 μl serum free DMEM/well. The solutions are gently mixed and incubated for 15-30 min at room temperature. Cells are then washed with 0.5 ml PBS and 400 μl of serum free media and then mixed with the transfection media and added to the cells. The cells are incubated for 3-4 hrs at 37° C./5% CO₂ and then the transfection media is removed and replaced with 1 ml/well of regular growth media. On day 3 the cells are labeled with ³H-myo-inositol. Briefly, the media is removed and the cells are washed with 0.5 ml PBS. Then 0.5 ml inositol-free/serum free media (GIBCO BRL) are added/well with 0.25 μCi of ³H-myo-inositol/well and the cells incubated for 16-18 hrs overnight at 37° C./5% CO₂. On Day 4 the cells are washed with 0.5 ml PBS and 0.45 ml of assay medium is added containing inositol-free/serum free media 10 μM pargyline 10 mM lithium chloride or 0.4 ml of assay medium and 50 μl of 10× ketanserin (ket) to final concentration of 10 μM. The cells are then incubated for 30 min at 37° C. The cells are then washed with 0.5 ml PBS and 200 μl of fresh/ice cold stop solution (1M KOH; 18 mM Na-borate; 3.8 mM EDTA) is added to each well. The solution is kept on ice for 5-10 min (or until cells are lysed) and then neutralized by 200 μl of fresh/ice cold neutralization solution (7.5% HCL). The lysate is then transferred into 1.5 ml Eppendorf tubes and 1 ml of chloroform/methanol (1:2) is added/tube. The solution is vortexed for 15 sec and the upper phase is applied to a Biorad AG1-X8™ anion exchange resin (100-200 mesh). First, the resin is washed with water at 1:1.25 W/V and 0.9 ml of upper phase is loaded onto the column. The column is then washed with 10 ml of 5 mM myo-inositol and 10 ml of 5 mM Na-borate/60 mM Na-formate. The inositol tris phosphates are eluted into scintillation vials containing 10 ml of scintillation cocktail with 2 ml of 0.1 M formic acid/1 M ammonium formate. The columns are regenerated by washing with 10 ml of 0.1 M formic acid/3M ammonium formate and rinsed twice with dd H₂O and stored at 4° C. in water.

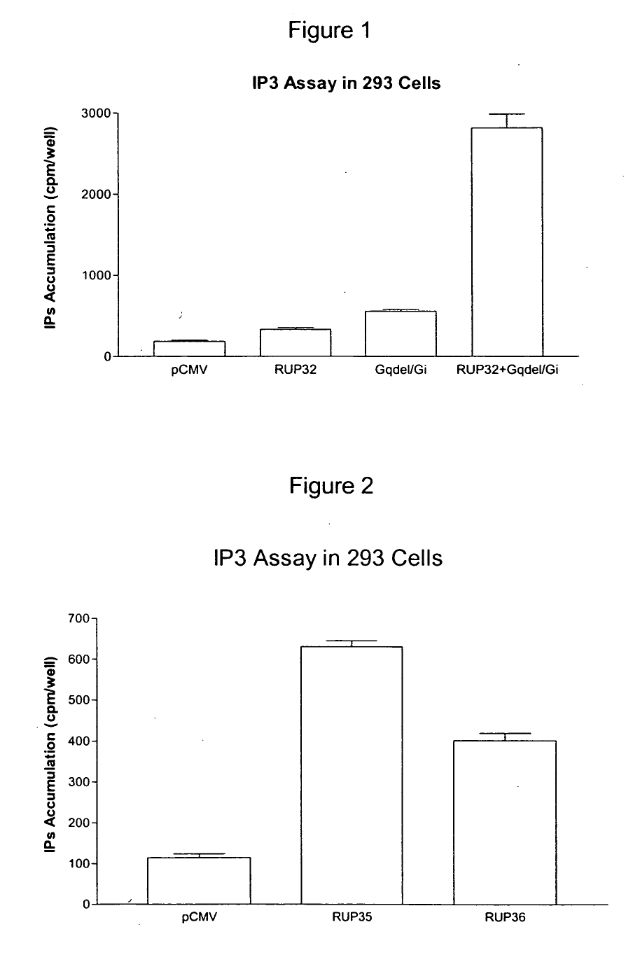

Reference is made to FIG. 1. In FIG. 1, 293 cells were transfected with G_{q} protein containing a six amino acid deletion, “G_{q}(del)”; G_{q} protein fused to a Gᵢ protein, “G_{q}(del)/Gᵢ”; endogenous RUP32; and RUP32 with G_{q}(del) (“RUP32+G_{q}(del)/Gᵢ”). The data indicate, based upon measuring IP₃ accumulation of RUP32 co-transfection of G_{q}(del)/Gᵢ, that RUP32 does not endogenously couple to G_{q} protein. However when RUP32 was co-transfected with G_{q}(del)/Gᵢ fusion protein, RUP32 was forced to couple to G_{q} protein. RUP27+G_{q}(del)/Gᵢ evidence about a nine (9) fold increase in IP₃ accumulation when compared to endogenous RUP32. This data demonstrates that the G_{q}(del)/Gᵢ Fusion Construct can be co-transfected with a GPCR and used to screen for agonists or inverse agonists.

Reference is made to FIG. 2. In FIG. 2, 293 cells were transfected with RUP35 and RUP36 receptor and compared to the control, pCMV. The data indicate that both RUP35 and RUP36 receptor are endogenously, constitutively active. RUP35 evidences about a six (6) fold increase in intracellular inositol phosphate accumulation when compared to pCMV and RUP36 evidences about a four (4) fold increase when compared to pCMV.

### Example 5

Fusion Protein Preparation

a. GPCR: Gₛ Fusion Construct

The design of the constitutively activated GPCR-G protein fusion construct can be accomplished as follows: both the 5′ and 3′ ends of the rat G protein Gₛα (long form; Itoh, H. et al., 83 PNAS 3776 (1986)) is engineered to include a HindIII (5′-AAGCTT-3′) sequence thereon. Following confirmation of the correct sequence (including the flanking HindIII sequences), the entire sequence is shuttled into pcDNA3.1(−) (Invitrogen, cat. no. V795-20) by subcloning using the HindIII restriction site of that vector. The correct orientation for the Gₛα (sequence will be determined after subcloning into pcDNA3.1(−). The modified pcDNA3.1(−) containing the rat Gₛα gene at HindIII sequence is then verified; this vector will then be available as a “universal” Gₛα protein vector. The pcDNA3.1(−) vector contains a variety of well-known restriction sites upstream of the HindIII site, thus beneficially providing the ability to insert, upstream of the Gₛ protein, the coding sequence of an endogenous, constitutively active GPCR. This same approach can be utilized to create other “universal” G protein vectors, and, of course, other commercially available or proprietary vectors known to the artisan can be utilized. In some embodiments, the important criteria is that the sequence for the GPCR be upstream and in-frame with that of the G protein.

Spacers in the restriction sites between the G protein and the GPCR are optional. The sense and anti-sense primers included the restriction sites for XbaI and EcoRV, respectively, such that spacers (attributed to the restriction sites) exist between the G protein and the GPCR.

PCR will then be utilized to secure the respective receptor sequences for fusion within the Gₛα universal vector disclosed above, using the following protocol for each: 100 ng cDNA for GPCR will be added to separate tubes containing 2 μl of each primer (sense and anti-sense), 3 μl of 10 mM dNTPs, 10 μl of 10× TaqPlus™ Precision buffer, 1 μl of TaqPlus™ Precision polymerase (Stratagene: #600211), and 80 μl of water. Reaction temperatures and cycle times for the GPCR will be as follows with cycle steps 2 through 4 were repeated 35 times: 94° C. for 1 min; 94° C. for 30 seconds; 62° C. for 20 sec; 72° C. 1 min 40 sec; and 72° C. 5 min. PCR products will be run on a 1% agarose gel and then purified. The purified products will be digested with XbaI and EcoRV and the desired inserts purified and ligated into the Gₛ universal vector at the respective restriction sites. The positive clones will be isolated following transformation and determined by restriction enzyme digestion; expression using 293 cells will be accomplished following the protocol set forth infra. Each positive clone for GPCR-Gₛ Fusion Protein will be sequenced to verify correctness.

b. G_{q}(6 Amino Acid Deletion)/Gᵢ Fusion Construct

The design of a G_{q}(del)/Gᵢ fusion construct was accomplished as follows: the N-terminal six (6) amino acids (amino acids 2 through 7), having the sequence of TLESIM (SEQ. ID. NO.:47) Gαq-subunit was deleted and the C-terminal five (5) amino acids, having the sequence EYNLV (SEQ. ID. NO.:48) was replaced with the corresponding amino acids of the Gαi Protein, having the sequence DCGLF (SEQ. ID. NO.:49). This fusion construct was obtained by PCR using the following primers: 5′-gatcAAGCTTCCATGGCGTGCTGCCTGAGCGAGG-3′ (SEQ. ID. NO.:50) and 5′-gatcGGATCCTTAGAACAGGCCGCAGTCCTTCAGGTTCAGCTGCAGGATGGTG-3′ (SEQ. ID. NO.:51) and Plasmid 63313 which contains the mouse Gαq-wild type version with a hemagglutinin tag as template. Nucleotides in lower caps are included as spacers.

TaqPlus® Precision DNA polymerase (Stratagene) was utilized for the amplification by the following cycles, with steps 2 through 4 repeated 35 times: 95° C. for 2 min; 95° C. for 20 sec; 56° C. for 20 sec; 72° C. for 2 min; and 72° C. for 7 min. The PCR product will be cloned into a pCRII-TOPO vector (Invitrogen) and sequenced using the ABI Big Dye Terminator kit (P.E. Biosystems). Inserts from a TOPO clone containing the sequence of the fusion construct will be shuttled into the expression vector pcDNA3.1 (+) at the HindIII/BamHI site by a 2 step cloning process.

### Example 6

Tissue Distribution of the Disclosed Human GPCRs: RT-PCR

RT-PCR was applied to confirm the expression and to determine the tissue distribution of several novel human GPCRs. Oligonucleotides utilized were GPCR-specific and the human multiple tissue cDNA panels (MTC, Clontech) as templates. Taq DNA polymerase (Stratagene) were utilized for the amplification in a 40 μl reaction according to the manufacturer's instructions. Twenty μl of the reaction will be loaded on a 1.5% agarose gel to analyze the RT-PCR products. Table E, below, lists the receptors, the cycle conditions and the primers utilized, and also lists exemplary diseases/disorders linked to the receptors.

TABLE E

| | Cycle | | | | |
|---|---|---|---|---|---|
| | Conditions | | | | |
| | Min (′), Sec (″) | | | | |
| | Cycles 2-4 | | | | |
| Receptor | repeated 35 | 5′ Primer | 3′ Primer | DNA | Tissue |
| Identifier | times | (SEQ. ID. NO.) | (SEQ. ID. NO) | Fragment | Expression |
| hRUP28 | 94° C. for 5 min; | GTCCTCACTG | CTGCGTCCAC | 710 bp | heart; kidney; |
| | 94° C. for 30 sec; | GTGGCCATGT | CAGAGTCACG | | liver, lung and |
| | 58° C. for 30 sec, | ACTCC | TCTCC | | pancreas |
| | 72° C. for 1 min, and | (52) | (53) | | |
| | 72° C. for 7 min | | | | |
| hRUP29 | 94° C. for 5 min; | CTTGGATGTT | GTTTGTGGCT | 690 bp | leukocyte and |
| | 94° C. for 30 sec; | TGGGCTGCCC | AACGGCACAA | | ovary |
| | 58° C. for 30 sec, | TTCTGC | AACACAATTC | | |
| | 72° C. for 1 min, and | (54) | C | | |
| | 72° C. for 7 min | | (55) | | |
| hRUP30 | 94° C. for 2 min; | CTGCTCACGG | GTGGCCATGA | 690 bp | pancreas |
| | 94° C. for 15 sec; | TTGACCGCTA | GCCACCCTGA | | |
| | 58° C. for 20 sec, | CACTGC | GCTCC | | |
| | 72° C. for 1 min, and | (56) | (57) | | |
| | 72° C. for 19 min | | | | |
| hRUP31 | 95° C. for 4 min; | CTTCTTCTCC | CCAAATCAGT | 516 bp | colon, lung, pancreas |
| | 95° C. for 1 min; | GACGTCAAGG | GTGCAAATCG | | thymus; cerebral cortex |
| | 52° C. for 30 sec, | (58) | (59) | | hippocampus of brain, and |
| | 72° C. for 1 min, and | | | | fat cells |
| | 72° C. for 7 min | | | | |
| hRUP32 | 95° C. for 4 min; | TGAATGGGTC | CAACGGTCTG | 527 bp | thymus |
| | 95° C. for 1 min; | CTGTGTGAAA | ACAACCTCCT | | |
| | 52° C. for 30 sec, | (60) | (61) | | |
| | 72° C. for 1 min, and | | | | |
| | 72° C. for 7 min | | | | |
| hRUP34 | 95° C. for 4 min; | TTGCTGTGAT | CAGGAAGCCC | 534 bp | peripheral blood |
| | 95° C. for 1 min; | GTGGCATTTT | ATAAAGGCAT | | leukocyte (“PBL”), |
| | 52° C. for 30 sec, | G | CAA | | prostate and |
| | 72° C. for 1 min, and | (62) | (63) | | kidney |
| | 72° C. for 7 min | | | | |
| hRUP35 | 95° C. for 4 min; | ACATCACCTG | CCAGCATCTT | 557 bp | thalamus |
| | 95° C. for 1 min; | CTTCCTGACC | GATGCAGTGT | | |
| | 52° C. for 30 sec, | (64) | (65) | | |
| | 72° C. for 1 min, and | | | | |
| | 72° C. for 7 min | | | | |
| hRUP37 | 95° C. for 4 min; | CCATCTCCAA | GCTGTTAAGA | 517 bp | testis, cerebral |
| | 95° C. for 1 min; | AATCCTCAGT | GCGGACAGGA | | cortex and |
| | 52° C. for 30 sec, | C | AA | | hippocampus |
| | 72° C. for 1 min, and | (66) | (67) | | |
| | 72° C. for 7 min | | | | |

Diseases and disorders related to receptors located in these tissues or regions include, but are not limited to, cardiac disorders and diseases (e.g. thrombosis, myocardial infarction; atherosclerosis; cardiomyopathies); kidney disease/disorders (e.g., renal failure; renal tubular acidosis; renal glycosuria; nephrogenic diabetes insipidus; cystinuria; polycystic kidney disease); eosinophilia; leukocytosis; leukopenia; ovarian cancer; sexual dysfunction; polycystic ovarian syndrome; pancreatitis and pancreatic cancer; irritable bowel syndrome; colon cancer; Crohn's disease; ulcerative colitis; diverticulitis; Chronic Obstructive Pulmonary Disease (COPD); Cystic Fibrosis; pneumonia; pulmonary hypertension; tuberculosis and lung cancer; Parkinson's disease; movement disorders and ataxias; learning and memory disorders; eating disorders (e.g., anorexia; bulimia, etc.); obesity; cancers; thymoma; myasthenia gravis; circulatory disorders; prostate cancer; prostatitis; kidney disease/disorders (e.g., renal failure; renal tubular acidosis; renal glycosuria; nephrogenic diabetes insipidus; cystinuria; polycystic kidney disease); sensorimotor processing and arousal disorders; obsessive-compulsive disorders; testicular cancer; priapism; prostatitis; hernia; endocrine disorders; sexual dysfunction; allergies; depression; psychotic disorders; migraine; reflux; schizophrenia; ulcers; bronchospasm; epilepsy; prostatic hypertrophy; anxiety; rhinitis; angina; and glaucoma. Accordingly, the methods of the present invention may also be useful in the diagnosis and/or treatment of these and other diseases and disorders.

### Example 7

Protocol: Direct Identification of Inverse Agonists and Agonists

A. [³⁵S]GTPγS Assay

Although endogenous, constitutively active GPCRs have been used for the direct identification of candidate compounds as, e.g., inverse agonists, for reasons that are not altogether understood, intra-assay variation can become exacerbated. In some embodiments a GPCR Fusion Protein, as disclosed above, is also utilized with a non-endogenous, constitutively activated GPCR. When such a protein is used, intra-assay variation appears to be substantially stabilized, whereby an effective signal-to-noise ratio is obtained. This has the beneficial result of allowing for a more robust identification of candidate compounds. Thus, in some embodiments it is preferred that for direct identification, a GPCR Fusion Protein be used and that when utilized, the following assay protocols be utilized.

1. Membrane Preparation

Membranes comprising the constitutively active orphan GPCR Fusion Protein of interest and for use in the direct identification of candidate compounds as inverse agonists or agonists are preferably prepared as follows:

a. Materials

“Membrane Scrape Buffer” is comprised of 20 mM HEPES and 10 mM EDTA, pH 7.4; “Membrane Wash Buffer” is comprised of 20 mM HEPES and 0.1 mM EDTA, pH 7.4; “Binding Buffer” is comprised of 20 mM HEPES, 100 mM NaCl, and 10 mM MgCl₂, pH 7.4 b. Procedure

All materials will be kept on ice throughout the procedure. Firstly, the media will be aspirated from a confluent monolayer of cells, followed by rinse with 10 ml cold PBS, followed by aspiration. Thereafter, 5 ml of Membrane Scrape Buffer will be added to scrape cells; this will be followed by transfer of cellular extract into 50 ml centrifuge tubes (centrifuged at 20,000 rpm for 17 minutes at 4° C.). Thereafter, the supernatant will be aspirated and the pellet will be resuspended in 30 ml Membrane Wash Buffer followed by centrifugation at 20,000 rpm for 17 minutes at 4° C. The supernatant will then be aspirated and the pellet resuspended in Binding Buffer. The resuspended pellet will then be homogenized using a Brinkman Polytron™ homogenizer (15-20 second bursts until the material is in suspension). This is referred to herein as “Membrane Protein”.

2. Bradford Protein Assay

Following the homogenization, protein concentration of the membranes will be determined, for example, using the Bradford Protein Assay (protein can be diluted to about 1.5 mg/ml, aliquoted and frozen (−80° C.) for later use; when frozen, protocol for use will be as follows: on the day of the assay, frozen Membrane Protein is thawed at room temperature, followed by vortex and then homogenized with a Polytron at about 12×1,000 rpm for about 5-10 seconds; it was noted that for multiple preparations, the homogenizer is thoroughly cleaned between homogenization of different preparations).

a. Materials

Binding Buffer (as discussed above); Bradford Dye Reagent; Bradford Protein Standard will be utilized, following manufacturer instructions (Biorad, cat. no. 500-0006).

b. Procedure

Duplicate tubes will be prepared, one including the membrane, and one as a control “blank”. Each contains 800 μl Binding Buffer. Thereafter, 10 μl of Bradford Protein Standard (1 mg/ml) will be added to each tube, and 10 μl of membrane Protein will then be added to just one tube (not the blank). Thereafter, 200 μl of Bradford Dye Reagent will be added to each tube, followed by vortexing. After five minutes, the tubes will be re-vortexed and the material therein will be transferred to cuvettes. The cuvettes will then be read using a CECIL 3041 spectrophotometer, at wavelength 595.

3. Direct Identification Assay

a. Materials

GDP Buffer consisted of 37.5 ml Binding Buffer and 2 mg GDP (Sigma, cat. no. G-7127), followed by a series of dilutions in Binding Buffer to obtain 0.2 μM GDP (final concentration of GDP in each well was 0.1 μM GDP); each well comprising a candidate compound, has a final volume of 200 μl consisting of 100 μl GDP Buffer (final concentration, 0.1 μM GDP), 50 μl Membrane Protein in Binding Buffer, and 50 μl [³⁵S]GTPγS (0.6 nM) in Binding Buffer (2.5 μl [³⁵S]GTPγS per 10 ml Binding Buffer).

b. Procedure

Candidate compounds will be preferably screened using a 96-well plate format (these can be frozen at −80° C.). Membrane Protein (or membranes with expression vector excluding the GPCR Fusion Protein, as control), will be homogenized briefly until in suspension. Protein concentration will then be determined using, for example, the Bradford Protein Assay set forth above. Membrane Protein (and controls) will then be diluted to 0.25 mg/ml in Binding Buffer (final assay concentration, 12.5 μg/well). Thereafter, 100 μl GDP Buffer is added to each well of a Wallac Scintistrip™ (Wallac). A 5 μl pin-tool will then be used to transfer 5 μl of a candidate compound into such well (i.e., 5 μl in total assay volume of 200 μl is a 1:40 ratio such that the final screening concentration of the candidate compound is 10 μM). Again, to avoid contamination, after each transfer step the pin tool is rinsed in three reservoirs comprising water (1×), ethanol (1×) and water (2×)—excess liquid is shaken from the tool after each rinse and the tool is dried with paper and Kim wipes. Thereafter, 50 μl of Membrane Protein will be added to each well (a control well comprising membranes without the GPCR Fusion Protein was also utilized), and pre-incubated for 5-10 minutes at room temperature. Thereafter, 50 μl of [³⁵S]GTPγS (0.6 nM) in Binding Buffer will be added to each well, followed by incubation on a shaker for 60 minutes at room temperature (again, in this example, plates were covered with foil). The assay will be stopped by spinning the plates at 4000 RPM for 15 minutes at 22° C. The plates will then be aspirated with an 8 channel manifold and sealed with plate covers. The plates will then be read on a Wallac 1450 using setting “Prot. #37” (as per manufacturer's instructions).

B. Cyclic AMP Assay

Another assay approach to directly identify candidate compound will be accomplished utilizing a cyclase-based assay. In addition to direct identification, this assay approach can be utilized as an independent approach to provide confirmation of the results from the [³⁵S]GTPγS approach as set forth above.

A modified Flash Plate™ Adenylyl Cyclase kit (New England Nuclear; Cat. No. SMP004A) will be preferably utilized for direct identification of candidate compounds as inverse agonists and agonists to GPCRs in accordance with the following protocol.

Transfected cells will be harvested approximately three days after transfection. Membranes will be prepared by homogenization of suspended cells in buffer containing 20 mM HEPES, pH 7.4 and 10 mM MgCl₂. Homogenization will be performed on ice using a Brinkman Polytron™ for approximately 10 seconds. The resulting homogenate will be centrifuged at 49,000×g for 15 minutes at 4° C. The resulting pellet will then be resuspended in buffer containing 20 mM HEPES, pH 7.4 and 0.1 mM EDTA, homogenized for 10 seconds, followed by centrifugation at 49,000×g for 15 minutes at 4° C. The resulting pellet will then be stored at −80° C. until utilized. On the day of direct identification screening, the membrane pellet will slowly be thawed at room temperature, resuspended in buffer containing 20 mM HEPES, pH 7.4 and 10 mM MgCl₂, to yield a final protein concentration of 0.60 mg/ml (the resuspended membranes will be placed on ice until use). cAMP standards and Detection Buffer (comprising 2 μCi of tracer [¹²⁵I cAMP (100 μl] to 11 ml Detection Buffer) will be prepared and maintained in accordance with the manufacturer's instructions. Assay Buffer will be prepared fresh for screening and contain 20 mM HEPES, pH 7.4, 10 mM MgCl₂, 20 mM phosphocreatine (Sigma), 0.1 units/ml creatine phosphokinase (Sigma), 50 μM GTP (Sigma), and 0.2 mM ATP (Sigma); Assay Buffer will be stored on ice until utilized.

Candidate compounds identified as per above (if frozen, thawed at room temperature) will be added, preferably, to 96-well plate wells (3 μl/well; 12 M final assay concentration), together with 40 μl Membrane Protein (30 μg/well) and 50 μl of Assay Buffer. This admixture will be incubated for 30 minutes at room temperature, with gentle shaking.

Following the incubation, 100 μl of Detection Buffer will be added to each well, followed by incubation for 2-24 hours. Plates will then be counted in a Wallac MicroBeta™ plate reader using “Prot. #31” (as per manufacturer instructions).

C. Melanophore Screening Assay

A method for identifying candidate agonists or inverse agonists for a GPCR can be preformed by introducing tests cells of a pigment cell line capable of dispersing or aggregating their pigment in response to a specific stimulus and expressing an exogenous clone coding for the GCPR. A stimulant, e.g., light, sets an initial state of pigment disposition wherein the pigment is aggregated within the test cells if activation of the GPCR induces pigment dispersion. However, stimulating the cell with a stimulant to set an initial state of pigment disposition wherein the pigment is dispersed if activation of the GPCR induces pigment aggregation. The tests cells are then contacted with chemical compounds, and it is determined whether the pigment disposition in the cells changed from the initial state of pigment disposition. Dispersion of pigments cells due to the candidate compound coupling to the GPCR will appear dark on a petri dish, while aggregation of pigments cells will appear light.

Materials and methods will be followed according to the disclosure of U.S. Pat. No. 5,462,856 and U.S. Pat. No. 6,051,386, each of which are incorporated by reference.

Although a variety of expression vectors are available to those in the art, for purposes of utilization for both the endogenous and non-endogenous human GPCRs, in some embodiments it is preferred that the vector utilized be pCMV. This vector was deposited with the American Type Culture Collection (ATCC) on Oct. 13, 1998 (10801 University Blvd., Manassas, Va. 20110-2209 USA) under the provisions of the Budapest Treaty for the International Recognition of the Deposit of Microorganisms for the Purpose of Patent Procedure. The DNA was tested by the ATCC and determined to be viable. The ATCC has assigned the following deposit number to pCMV: ATCC #203351.

References cited throughout this patent document, including co-pending and related patent applications, unless otherwise indicated, are fully incorporated herein by reference. Modifications and extension of the disclosed inventions that are within the purview of the skilled artisan are encompassed within the above disclosure and the claims that follow.

What is claimed is:

1. A G protein-coupled receptor encoded by an amino acid sequence of SEQ. ID. NO.:2.

2. A non-endogenous, constitutively activated version of the G protein-coupled receptor of claim 1.

3. A plasmid comprising a vector and the cDNA of SEQ. ID. NO.:1.

4. A host cell comprising the plasmid of claim 3.

5. A G protein-coupled receptor encoded by an amino acid sequence of SEQ. ID. NO.:4.

6. A non-endogenous, constitutively activated version of the G protein-coupled receptor of claim 5.

7. A plasmid comprising a vector and the cDNA of SEQ. ID. NO.:3.

8. A host cell comprising the plasmid of claim 7.

9. A G protein-coupled receptor encoded by an amino acid sequence of SEQ. ID. NO.:6.

10. A non-endogenous, constitutively activated version of the G protein-coupled receptor of claim 9.

11. A plasmid comprising a vector and the cDNA of SEQ. ID. NO.:5.

12. A host cell comprising the plasmid of claim 11.

13. A G protein-coupled receptor encoded by an amino acid sequence of SEQ. ID. NO.:8.

14. A non-endogenous, constitutively activated version of the G protein-coupled receptor of claim 13.

15. A plasmid comprising a vector and the cDNA of SEQ. ID. NO.:7.

16. A host cell comprising the plasmid of claim 15.

17. A G protein-coupled receptor encoded by an amino acid sequence of SEQ. ID. NO.: 10.

18. A non-endogenous, constitutively activated version of the G protein-coupled receptor of claim 17.

19. A plasmid comprising a vector and the cDNA of SEQ. ID. NO.:9.

20. A host cell comprising the plasmid of claim 19.

21. A G protein-coupled receptor encoded by an amino acid sequence of SEQ. ID. NO.: 12.

22. A non-endogenous, constitutively activated version of the G protein-coupled receptor of claim 21.

23. A plasmid comprising a vector and the cDNA of SEQ. ID. NO.:11.

24. A host cell comprising the plasmid of claim 23.

25. A G protein-coupled receptor encoded by an amino acid sequence of SEQ. ID. NO.:14.

26. A non-endogenous, constitutively activated version of the G protein-coupled receptor of claim 25.

27. A plasmid comprising a vector and the cDNA of SEQ. ID. NO.:13.

28. A host cell comprising the plasmid of claim 27.

29. A G protein-coupled receptor encoded by an amino acid sequence of SEQ. ID. NO.:16.

30. A non-endogenous, constitutively activated version of the G protein-coupled receptor of claim 29.

31. A plasmid comprising a vector and the cDNA of SEQ. ID. NO.:15.

32. A host cell comprising the plasmid of claim 31.

33. A G protein-coupled receptor encoded by an amino acid sequence of SEQ. ID. NO.: 18.

34. A non-endogenous, constitutively activated version of the G protein-coupled receptor of claim 33.

35. A plasmid comprising a vector and the cDNA of SEQ. ID. NO.:17.

36. A host cell comprising the plasmid of claim 35.

37. A G protein-coupled receptor encoded by an amino acid sequence of SEQ. ID. NO.:20.

38. A non-endogenous, constitutively activated version of the G protein-coupled receptor of claim 37.

39. A plasmid comprising a vector and the cDNA of SED. ID. NO.: 19.

40. A host cell comprising the plasmid of claim 39.


SEQUENCE LISTING (67 sequences)

SEQ ID NO: 1 - DNA, 1002 bp, Artificial
  Novel Sequence
atgtggagctgcagctggttcaacggcacagggctggtggaggagctgcctgcctgccaggacctgcagctggggctgtcactgttgtcgctgctgggcctggtggtgggcgtgccagtgggcctgtgctacaacgccctgctggtgctggccaacctacacagcaaggccagcatgaccatgccggacgtgtactttgtcaacatggcagtggcaggcctggtgctcagcgccctggcccctgtgcacctgctcggccccccgagctcccggtgggcgctgtggagtgtgggcggcgaagtccacgtggcactgcagatccccttcaatgtgtcctcactggtggccatgtactccaccgccctgctgagcctcgaccactacatcgagcgtgcactgccgcggacctacatggccagcgtgtacaacacgcggcacgtgtgcggcttcgtgtggggtggcgcgctgctgaccagcttctcctcgctgctcttctacatctgcagccatgtgtccacccgcgcgctagagtgcgccaagatgcagaacgcagaagctgccgacgccacgctggtgttcatcggctacgtggtgccagcactggccaccctctacgcgctggtgctactctcccgcgtccgcagggaggacacgcccctggaccgggacacgggccggctggagccctcggcacacaggctgctggtggccaccgtgtgcacgcagtttgggctctggacgccacactatctgatcctgctggggcacacggtcatcatctcgcgagggaagcccgtggacgcacactacctggggctactgcactttgtgaaggatttctccaaactcctggccttctccagcagctttgtgacaccacttctctaccgctacatgaaccagagcttccccagcaagctccaacggctgatgaaaaagctgccctgcggggaccggcactgctccccggaccacatgggggtgcagcaggtgctggcgtag

SEQ ID NO: 2 - protein, 333 aa, Artificial
  Novel Sequence
MWSCSWFNGTGLVEELPACQDLQLGLSLLSLLGLVVGVPVGLCYNALLVLANLHSKASMTMPDVYFVNMAVAGLVLSALAPVHLLGPPSSRWALWSVGGEVHVALQIPFNVSSLVAMYSTALLSLDHYIERALPRTYMASVYNTRHVCGFVWGGALLTSFSSLLFYICSHVSTRALECAKMQNAEAADATLVFIGYVVPALATLYALVLLSRVRREDTPLDRDTGRLEPSAHRLLVATVCTQFGLWTPHYLILLGHTVIISRGKPVDAHYLGLLHFVKDFSKLLAFSSSFVTPLLYRYMNQSFPSKLQRLMKKLPCGDRHCSPDHMGVQQVLA

SEQ ID NO: 3 - DNA, 918 bp, Homo sapien
atgcctggccacaatacctccaggaattcctcttgcgatcctatagtgacaccccacttaatcagcctctacttcatagtgcttattggcgggctggtgggtgtcatttccattcttttcctcctggtgaaaatgaacacccggtcagtgaccaccatggcggtcattaacttggtggtggtccacagcgtttttctgctgacagtgccatttcgcttgacctacctcatcaagaagacttggatgtttgggctgcccttctgcaaatttgtgagtgccatgctgcacatccacatgtacctcacgttcctattctatgtggtgatcctggtcaccagatacctcatcttcttcaagtgcaaagacaaagtggaattctacagaaaactgcatgctgtggctgccagtgctggcatgtggacgctggtgattgtcattgtggtacccctggttgtctcccggtatggaatccatgaggaatacaatgaggagcactgttttaaatttcacaaagagcttgcttacacatatgtgaaaatcatcaactatatgatagtcatttttgtcatagccgttgctgtgattctgttggtcttccaggtcttcatcattatgttgatggtgcagaagctacgccactctttactatcccaccaggagttctgggctcagctgaaaaacctattttttataggggtcatccttgtttgtttccttccctaccagttctttaggatctattacttgaatgttgtgacgcattccaatgcctgtaacagcaaggttgcattttataacgaaatcttcttgagtgtaacagcaattagctgctatgatttgcttctctttgtctttgggggaagccattggtttaagcaaaagataattggcttatggaattgtgttttgtgccgttag

SEQ ID NO: 4 - protein, 305 aa, Homo sapien
MPGHNTSRNSSCDPIVTPHLISLYFIVLIGGLVGVISILFLLVKMNTRSVTTMAVINLVVVHSVFLLTVPFRLTYLIKKTWMFGLPFCKFVSAMLHIHMYLTFLFYVVILVTRYLIFFKCKDKVEFYRKLHAVAASAGMWTLVIVIVVPLVVSRYGIHEEYNEEHCFKFHKELAYTYVKIINYMIVIFVIAVAVILLVFQVFIIMLMVQKLRHSLLSHQEFWAQLKNLFFIGVILVCFLPYQFFRIYYLNVVTHSNACNSKVAFYNEIFLSVTAISCYDLLLFVFGGSHWFKQKIIGLWNCVLCR

SEQ ID NO: 5 - DNA, 1125 bp, Artificial
  Novel Sequence
atgctgacagggagctgcggggaccctcagaaaaagccacaggtgacccaggactcagggccccagagcatggggcttgagggacgagagacagctggccagccacgagtgaccctgctgcccacgcccaacgtcagcgggctgagccaggagtttgaaagccactggccagagatcgcagagaggtccccgtgtgtggctggcgtcatccctgtcatctactacagtgtcctgctgggcttggggctgcctgtcagcctcctgaccgcagtggccctggcgcgccttgccaccaggaccaggaggccctcctactactaccttctggcgctcacagcctcggatatcatcatccaggtggtcatcgtgttcgcgggcttcctcctgcagggagcagtgctggcccgccaggtgccccaggctgtggtgcgcacggccaacatcctggagtttgctgccaaccacgcctcagtctggatcgccatcctgctcacggttgaccgctacactgccctgtgccaccccctgcaccatcgggccgcctcgtccccaggccggacccgccgggccattgctgctgtcctgagtgctgccctgttgaccggcatccccttctactggtggctggacatgtggagagacaccgactcacccagaacactggacgaggtcctcaagtgggctcactgtctcactgtctatttcatcccttgtggcgtgttcctggtcaccaactcggccatcatccaccggctacggaggaggggccggagtgggctgcagccccgggtgggcaagagcacagccatcctcctgggcatcaccacactgttcaccctcctgtgggcgccccgggtcttcgtcatgctctaccacatgtacgtggcccctgtccaccgggactggagggtccacctggccttggatgtggccaatatggtggccatgctccacacggcagccaacttcggcctctactgctttgtcagcaagactttccgggccactgtccgacaggtcatccacgatgcctacctgccctgcactttggcatcacagccagagggcatggcggcgaagcctgtgatggagcctccgggactccccacaggggcagaagtgtag

SEQ ID NO: 6 - protein, 374 aa, Artificial [sequence omitted]

SEQ ID NO: 7 - DNA, 1086 bp, Artificial
  Novel Sequencw
atgtccactgaatgcgcgcgggcagcgggcgacgcgcccttgcgcagcctggagcaagccaaccgcacccgctttcccttcttctccgacgtcaagggcgaccaccggctggtgctggccgcggtggagacaaccgtgctggtgctcatctttgcagtgtcgctgctgggcaacgtgtgcgccctggtgctggtggcgcgccgacgacgccgcggcgcgactgcctgcctggtactcaacctcttctgcgcggacctgctcttcatcagcgctatccctctggtgctggccgtgcgctggactgaggcctggctgctgggccccgttgcctgccacctgctcttctacgtgatgaccctgagcggcagcgtcaccatcctcacgctggccgcggtcagcctggagcgcatggtgtgcatcgtgcacctgcagcgcggcgtgcggggtcctgggcggcgggcgcgggcagtgctgctggcgctcatatggggctattcggcggtcgccgctctgcctctatgcgtcttcttccgagtcgtcccgcaacggctccccggcgccgaccaggaaatttcgatttgcacactgatttggcccaccattcctggagagatctcgtgggatgtctcttttgttactttgaacttcttggtgccaggactggtcattgtgatcagttactccaaaattttacagatcacaaaggcatcaaggaagaggctcacggtaagcctggcctactcggagagccaccagatccgcgtgtcccagcaggacttccggctcttccgcaccctcttcctcctcatggtctccttcttcatcatgtggagccccatcatcatcaccatcctcctcatcctgatccagaacttcaagcaagacctggtcatctggccgtccctcttcttctgggtggtggccttcacatttgctaattcagccctaaaccccatcctctacaacatgacactgtgcaggaatgagtggaagaaaattttttgctgcttctggttcccagaaaagggagccattttaacagacacatctgtcaaaagaaatgacttgtcgattatttctggctaa

SEQ ID NO: 8 - protein, 361 aa, Artificial
  Novel Sequence
MSTECARAAGDAPLRSLEQANRTRFPFFSDVKGDHRLVLAAVETTVLVLIFAVSLLGNVCALVLVARRRRRGATACLVLNLFCADLLFISAIPLVLAVRWTEAWLLGPVACHLLFYVMTLSGSVTILTLAAVSLERMVCIVHLQRGVRGPGRRARAVLLALIWGYSAVAALPLCVFFRVVPQRLPGADQEISICTLIWPTIPGEISWDVSFVTLNFLVPGLVIVISYSKILQITKASRKRLTVSLAYSESHQIRVSQQDFRLFRTLFLLMVSFFIMWSPIIITILLILIQNFKQDLVIWPSLFFWVVAFTFANSALNPILYNMTLCRNEWKKIFCCFWFPEKGAILTDTSVKRNDLSIISG

SEQ ID NO: 9 - DNA, 1038 bp, Artificial
  Novel Sequence
atgagcagcaattcatccctgctggtggctgtgcagctgtgctacgcgaacgtgaatgggtcctgtgtgaaaatccccttctcgccgggatcccgggtgattctgtacatagtgtttggctttggggctgtgctggctgtgtttggaaacctcctggtgatgatttcaatcctccatttcaagcagctgcactctccgaccaattttctcgttgcctctctggcctgcgctgatttcttggtgggtgtgactgtgatgcccttcagcatggtcaggacggtggagagctgctggtattttgggaggagtttttgtactttccacacctgctgtgatgtggcattttgttactcttctctctttcacttgtgcttcatctccatcgacaggtacattgcggttactgaccccctggtctatcctaccaagttcaccgtatctgtgtcaggaatttgcatcagcgtgtcctggatcctgcccctcatgtacagcggtgctgtgttctacacaggtgtctatgacgatgggctggaggaattatctgatgccctaaactgtataggaggttgtcagaccgttgtaaatcaaaactgggtgttgacagattttctatccttctttatacctacctttattatgataattctgtatggtaacatatttcttgtggctagacgacaggcgaaaaagatagaaaatactggtagcaagacagaatcatcctcagagagttacaaagccagagtggccaggagagagagaaaagcagctaaaaccctgggggtcacagtggtagcatttatgatttcatggttaccatatagcattgattcattaattgatgcctttatgggctttataacccctgcctgtatttatgagatttgctgttggtgtgcttattataactcagccatgaatcctttgatttatgctttattttacccatggtttaggaaagcaataaaagttattgtaactggtcaggttttaaagaacagttcagcaaccatgaatttgttttctgaacatatataa

SEQ ID NO: 10 - protein, 345 aa, Artificial
  Novel Sequence
MSSNSSLLVAVQLCYANVNGSCVKIPFSPGSRVILYIVFGFGAVLAVFGNLLVMISILHFKQLHSPTNFLVASLACADFLVGVTVMPFSMVRTVESCWYFGRSFCTFHTCCDVAFCYSSLFHLCFISIDRYIAVTDPLVYPTKFTVSVSGICISVSWILPLMYSGAVFYTGVYDDGLEELSDALNCIGGCQTVVNQNWVLTDFLSFFIPTFIMIILYGNIFLVARRQAKKIENTGSKTESSSESYKARVARRERKAAKTLGVTVVAFMISWLPYSIDSLIDAFMGFITPACIYEICCWCAYYNSAMNPLIYALFYPWFRKAIKVIVTGQVLKNSSATMNLFSEHI

SEQ ID NO: 11 - DNA, 1020 bp, Artificial
  Novel Sequence
atgatgcccttttgccacaatataattaatatttcctgtgtgaaaaacaactggtcaaatgatgtccgtgcttccctgtacagtttaatggtgctcataattctgaccacactcgttggcaatctgatagttattgtttctatatcacacttcaaacaacttcataccccaacaaattggctcattcattccatggccactgtggactttcttctggggtgtctggtcatgccttacagtatggtgagatctgctgagcactgttggtattttggagaagtcttctgtaaaattcacacaagcaccgacattatgctgagctcagcctccattttccatttgtctttcatctccattgaccgctactatgctgtgtgtgatccactgagatataaagccaagatgaatatcttggttatttgtgtgatgatcttcattagttggagtgtccctgctgtttttgcatttggaatgatctttctggagctaaacttcaaaggcgctgaagagatatattacaaacatgttcactgcagaggaggttgctctgtcttctttagcaaaatatctggggtactgacctttatgacttctttttatatacctggatctattatgttatgtgtctattacagaatatatcttatcgctaaagaacaggcaagattaattagtgatgccaatcagaagctccaaattggattggaaatgaaaaatggaatttcacaaagcaaagaaaggaaagctgtgaagacattggggattgtgatgggagttttcctaatatgctggtgccctttctttatctgtacagtcatggacccttttcttcactacattattccacctactttgaatgatgtattgatttggtttggctacttgaactctacatttaatccaatggtttatgcatttttctatccttggtttagaaaagcactgaagatgatgctgtttggtaaaattttccaaaaagattcatccaggtgtaaattatttttggaattgagttcatag

SEQ ID NO: 12 - protein, 339 aa, Artificial
  Novel Sequence
MMPFCHNIINISCVKNNWSNDVRASLYSLMVLIILTTLVGNLIVIVSISHFKQLHTPTNWLIHSMATVDFLLGCLVMPYSMVRSAEHCWYFGEVFCKIHTSTDIMLSSASIFHLSFISIDRYYAVCDPLRYKAKMNILVICVMIFISWSVPAVFAFGMIFLELNFKGAEEIYYKHVHCRGGCSVFFSKISGVLTFMTSFYIPGSIMLCVYYRIYLIAKEQARLISDANQKLQIGLEMKNGISQSKERKAVKTLGIVMGVFLICWCPFFICTVMDPFLHYIIPPTLNDVLIWFGYLNSTFNPMVYAFFYPWFRKALKMMLFGKIFQKDSSRCKLFLELSS

SEQ ID NO: 13 - DNA, 1029 bp, Artificial
  Novel Sequence
atgaccagcaatttttcccaacctgttgtgcagctttgctatgaggatgtgaatggatcttgtattgaaactccctattctcctgggtcccgggtaattctgtacacggcgtttagctttgggtctttgctggctgtatttggaaatctcttagtaatgacttctgttcttcattttaagcagctgcactctccaaccaattttctcattgcctctctggcctgtgctgacttcttggtaggtgtgactgtgatgcttttcagcatggtcaggacggtggagagctgctggtattttggagccaaattttgtactcttcacagttgctgtgatgtggcattttgttactcttctgtcctccacttgtgcttcatctgcatcgacaggtacattgtggttactgatcccctggtctatgctaccaagttcaccgtgtctgtgtcgggaatttgcatcagcgtgtcctggattctgcctctcacgtacagcggtgctgtgttctacacaggtgtcaatgatgatgggctggaggaattagtaagtgctctcaactgcgtaggtggctgtcaaattattgtaagtcaaggctgggtgttgatagattttctgttattcttcatacctacccttgttatgataattctttacagtaagatttttcttatagctaaacaacaagctataaaaattgaaactactagtagcaaagtagaatcatcctcagagagttataaaatcagagtggccaagagagagaggaaagcagctaaaaccctgggggtcacggtactagcatttgttatttcatggttaccgtatacagttgatatattaattgatgcctttatgggcttcctgacccctgcctatatctatgaaatttgctgttggagtgcttattataactcagccatgaatcctttgatttatgctctattttatccttggtttaggaaagccataaaacttattttaagtggagatgttttaaaggctagttcatcaaccattagtttatttttagaataa

SEQ ID NO: 14 - protein, 342 aa, Artificial
  Novel Sequence
MTSNFSQPVVQLCYEDVNGSCIETPYSPGSRVILYTAFSFGSLLAVFGNLLVMTSVLHFKQLHSPTNFLIASLACADFLVGVTVMLFSMVRTVESCWYFGAKFCTLHSCCDVAFCYSSVLHLCFICIDRYIVVTDPLVYATKFTVSVSGICISVSWILPLTYSGAVFYTGVNDDGLEELVSALNCVGGCQIIVSQGWVLIDFLLFFIPTLVMIILYSKIFLIAKQQAIKIETTSSKVESSSESYKIRVAKRERKAAKTLGVTVLAFVISWLPYTVDILIDAFMGFLTPAYIYEICCWSAYYNSAMNPLIYALFYPWFRKAIKLILSGDVLKASSSTISLFLE

SEQ ID NO: 15 - DNA, 1062 bp, Artificial
  Novel Sequence
atggagcacacgcacgcccacctcgcagccaacagctcgctgtcttggtggtcccccggctcggcctgcggcttgggtttcgtgcccgtggtctactacagcctcttgctgtgcctcggtttaccagcaaatatcttgacagtgatcatcctctcccagctggtggcaagaagacagaagtcctcctacaactatctcttggcactcgctgctgccgacatcttggtcctctttttcatagtgtttgtggacttcctgttggaagatttcatcttgaacatgcagatgcctcaggtccccgacaagatcatagaagtgctggaattctcatccatccacacctccatatggattactgtaccgttaaccattgacaggtatatcgctgtctgccacccgctcaagtaccacacggtctcatacccagcccgcacccggaaagtcattgtaagtgtttacatcacctgcttcctgaccagcatcccctattactggtggcccaacatctggactgaagactacatcagcacctctgtgcatcacgtcctcatctggatccactgcttcaccgtctacctggtgccctgctccatcttcttcatcttgaactcaatcattgtgtacaagctcaggaggaagagcaattttcgtctccgtggctactccacggggaagaccaccgccatcttgttcaccattacctccatctttgccacactttgggccccccgcatcatcatgattctttaccacctctatggggcgcccatccagaaccgctggctggtgcacatcatgtccgacattgccaacatgctagcccttctgaacacagccatcaacttcttcctctactgcttcatcagcaagcggttccgcaccatggcagccgccacgctcaaggctttcttcaagtgccagaagcaacctgtacagttctacaccaatcataacttttccataacaagtagcccctggatctcgccggcaaactcacactgcatcaagatgctggtgtaccagtatgacaaaaatggaaaacctataaaagtatccccgtga

SEQ ID NO: 16 - protein, 353 aa, Artificial
  Sequence
MEHTHAHLAANSSLSWWSPGSACGLGFVPVVYYSLLLCLGLPANILTVIILSQLVARRQKSSYNYLLALAAADILVLFFIVFVDFLLEDFILNMQMPQVPDKIIEVLEFSSIHTSIWITVPLTIDRYIAVCHPLKYHTVSYPARTRKVIVSVYITCFLTSIPYYWWPNIWTEDYISTSVHHVLIWIHCFTVYLVPCSIFFILNSIIVYKLRRKSNFRLRGYSTGKTTAILFTITSIFATLWAPRIIMILYHLYGAPIQNRWLVHIMSDIANMLALLNTAINFFLYCFISKRFRTMAAATLKAFFKCQKQPVQFYTNHNFSITSSPWISPANSHCIKMLVYQYDKNGKPIKVSP

SEQ ID NO: 17 - DNA, 969 bp, Artificial
  Novel Sequence
atggatccaaccgtcccagtcttcggtacaaaactgacaccaatcaacggacgtgaggagactccttgctacaatcagaccctgagcttcacggtgctgacgtgcatcatttcccttgtcggactgacaggaaacgcggtagtgctctggctcctgggctaccgcatgcgcaggaacgctgtctccatctacatcctcaacctggccgcagcagacttcctcttcctcagcttccagattatacgttcgccattacgcctcatcaatatcagccatctcatccgcaaaatcctcgtttctgtgatgacctttccctactttacaggcctgagtatgctgagcgccatcagcaccgagcgctgcctgtctgttctgtggcccatctggtaccgctgccgccgccccacacacctgtcagcggtcgtgtgtgtcctgctctggggcctgtccctgctgtttagtatgctggagtggaggttctgtgacttcctgtttagtggtgctgattctagttggtgtgaaacgtcagatttcatcccagtcgcgtggctgatttttttatgtgtggttctctgtgtttccagcctggtcctgctggtcaggatcctctgtggatcccggaagatgccgctgaccaggctgtacgtgaccatcctgctcacagtgctggtcttcctcctctgcggcctgcccttcggcattctgggggccctaatttacaggatgcacctgaatttggaagtcttatattgtcatgtttatctggtttgcatgtccctgtcctctctaaacagtagtgccaaccccatcatttacttcttcgtgggctcctttaggcagcgtcaaaataggcagaacctgaagctggttctccagagggctctgcaggacaagcctgaggtggataaaggtgaagggcagcttcctgaggaaagcctggagctgtcgggaagcagattggggccatga

SEQ ID NO: 18 - protein, 322 aa, Artificial
  Novel Sequence
MDPTVPVFGTKLTPINGREETPCYNQTLSFTVLTCIISLVGLTGNAVVLWLLGYRMRRNAVSIYILNLAAADFLFLSFQIIRSPLRLINISHLIRKILVSVMTFPYFTGLSMLSAISTERCLSVLWPIWYRCRRPTHLSAVVCVLLWGLSLLFSMLEWRFCDFLFSGADSSWCETSDFIPVAWLIFLCVVLCVSSLVLLVRILCGSRKMPLTRLYVTILLTVLVFLLCGLPFGILGALIYRMHLNLEVLYCHVYLVCMSLSSLNSSANPIIYFFVGSFRQRQNRQNLKLVLQRALQDKPEVDKGEGQLPEESLELSGSRLGP

SEQ ID NO: 19 - DNA, 969 bp, Artificial
  Novel Sequence
atggattcaaccatcccagtcttgggtacagaactgacaccaatcaacggacgtgaggagactccttgctacaagcagaccctgagcttcacggggctgacgtgcatcgtttcccttgtcgcgctgacaggaaacgcggttgtgctctggctcctgggctgccgcatgcgcaggaacgctgtctccatctacatcctcaacctggtcgcggccgacttcctcttccttagcggccacattatatgttcgccgttacgcctcatcaatatccgccatcccatctccaaaatcctcagtcctgtgatgacctttccctactttataggcctaagcatgctgagcgccatcagcaccgagcgctgcctgtccatcctgtggcccatctggtaccactgccgccgccccagatacctgtcatcggtcatgtgtgtcctgctctgggccctgtccctgctgcggagtatcctggagtggatgttctgtgacttcctgtttagtggtgctgattctgtttggtgtgaaacgtcagatttcattacaatcgcgtggctggtttttttatgtgtggttctctgtgggtccagcctggtcctgctggtcaggattctctgtggatcccggaagatgccgctgaccaggctgtacgtgaccatcctcctcacagtgctggtcttcctcctctgtggcctgccctttggcattcagtgggccctgttttccaggatccacctggattggaaagtcttattttgtcatgtgcatctagtttccattttcctgtccgctcttaacagcagtgccaaccccatcatttacttcttcgtgggctcctttaggcagcgtcaaaataggcagaacctgaagctggttctccagagggctctgcaggacacgcctgaggtggatgaaggtggagggtggcttcctcaggaaaccctggagctgtcgggaagcagattggagcagtga

SEQ ID NO: 20 - protein, 322 aa, Artificial
  Novel Sequence
MDSTIPVLGTELTPINGREETPCYKQTLSFTGLTCIVSLVALTGNAVVLWLLGCRMRRNAVSIYILNLVAADFLFLSGHIICSPLRLINIRHPISKILSPVMTFPYFIGLSMLSAISTERCLSILWPIWYHCRRPRYLSSVMCVLLWALSLLRSILEWMFCDFLFSGADSVWCETSDFITIAWLVFLCVVLCGSSLVLLVRILCGSRKMPLTRLYVTILLTVLVFLLCGLPFGIQWALFSRIHLDWKVLFCHVHLVSIFLSALNSSANPIIYFFVGSFRQRQNRQNLKLVLQRALQDTPEVDEGGGWLPQETLELSGSRLEQ

SEQ ID NO: 21 - DNA, 26 bp, Artificial
  Novel Sequence
cagagctctggtggccacctctgtcc

SEQ ID NO: 22 - DNA, 25 bp, Artificial
  Novel Sequence
ctgcgtccaccagagtcacgtctcc

SEQ ID NO: 23 - DNA, 26 bp, Artificial
  Novel Sequence
gtatgcctggccacaatacctccagg

SEQ ID NO: 24 - DNA, 31 bp, Artificial
  Novel Sequence
gtttgtggctaacggcacaaaacacaattcc

SEQ ID NO: 25 - DNA, 28 bp, Artificial
  Novel Sequence
ggtaccacaatgacaatcaccagcgtcc

SEQ ID NO: 26 - DNA, 29 bp, Artificial
  Novel Sequence
ggaacgtgaggtacatgtggatgtgcagc

SEQ ID NO: 27 - DNA, 27 bp, Artificial
  Novel Sequence
gcagtgtagcggtcaaccgtgagcagg

SEQ ID NO: 28 - DNA, 32 bp, Artificial
  Novel Sequence
tgagcaggatggcgatccagactgaggcgtgg

SEQ ID NO: 29 - DNA, 25 bp, Artificial
  Novel Sequence
gaggtacagctggcgatgctgacag

SEQ ID NO: 30 - DNA, 25 bp, Artificial
  Novel Sequence
gtggccatgagccaccctgagctcc

SEQ ID NO: 31 - DNA, 24 bp, Artificial
  Novel Sequence
ggaatgtccactgaatgcgcgcgg

SEQ ID NO: 32 - DNA, 25 bp, Artificial
  Novel Sequence
agctcgccaggtgtgagaaactcgg

SEQ ID NO: 33 - DNA, 30 bp, Artificial
  Novel Sequence
gcgttatgagcagcaattcatccctgctgg

SEQ ID NO: 34 - DNA, 28 bp, Artificial
  Novel Sequence
gtatcctgaacttcgtctatacaactgc

SEQ ID NO: 35 - DNA, 29 bp, Artificial
  Novel Sequence
ccctcaggaatgatgcccttttgccacaa

SEQ ID NO: 36 - DNA, 28 bp, Artificial
  Novel Sequence
atccatgtggttggtgcatgtggttcgt

SEQ ID NO: 37 - DNA, 29 bp, Artificial
  Novel Sequence
aaacaacaaacagcagaaccatgaccagc

SEQ ID NO: 38 - DNA, 30 bp, Artificial
  Novel Sequence
acatagagacaagtgacatgtgtgaaccac

SEQ ID NO: 39 - DNA, 26 bp, Artificial
  Novel Sequence
ggtatgagaccgtgtggtacttgagc

SEQ ID NO: 40 - DNA, 29 bp, Artificial
  Novel Sequence
gtggcagacagcgatatacctgtcaatgg

SEQ ID NO: 41 - DNA, 27 bp, Artificial
  Novel Sequence
gcgctcatggagcacacgcacgcccac

SEQ ID NO: 42 - DNA, 25 bp, Artificial
  Novel Sequence
gaggcagtagttgccacacctatgg

SEQ ID NO: 43 - DNA, 29 bp, Artificial
  Novel Sequence
catctggtttgtgttcccaggggcaccag

SEQ ID NO: 44 - DNA, 32 bp, Artificial
  Novel Sequence
gacagtgttgctctcaaagtcccgtctgactg

SEQ ID NO: 45 - DNA, 25 bp, Artificial
  Novel Sequence
ctgtttccagggtcatcagactggg

SEQ ID NO: 46 - DNA, 27 bp, Artificial
  Novel Sequence
gcagcattgctctcaaagtcctgtctg

SEQ ID NO: 47 - protein, 6 aa, Artificial
  Novel Sequence
TLESIM

SEQ ID NO: 48 - protein, 5 aa, Artificial
  Novel Sequence
EYNLV

SEQ ID NO: 49 - protein, 5 aa, Artificial
  Novel Sequence
DCGLF

SEQ ID NO: 50 - DNA, 34 bp, Artificial
  Novel Sequence
gatcaagcttccatggcgtgctgcctgagcgagg

SEQ ID NO: 51 - DNA, 53 bp, Artificial
  Novel Sequence
gatcggatccttagaacaggccgcagtccttcaggttcagctgcaggatggtg

SEQ ID NO: 52 - DNA, 25 bp, Artificial
  Novel Sequence
gtcctcactggtggccatgtactcc

SEQ ID NO: 53 - DNA, 25 bp, Artificial
  Novel Sequence
ctgcgtccaccagagtcacgtctcc

SEQ ID NO: 54 - DNA, 26 bp, Artificial
  Novel Sequence
cttggatgtttgggctgcccttctgc

SEQ ID NO: 55 - DNA, 31 bp, Artificial; identical to SEQ ID NO: 24
  Novel Sequence

SEQ ID NO: 56 - DNA, 26 bp, Artificial
  Novel Sequence
ctgctcacggttgaccgctacactgc

SEQ ID NO: 57 - DNA, 25 bp, Artificial
  Novel Sequence
gtggccatgagccaccctgagctcc

SEQ ID NO: 58 - DNA, 20 bp, Artificial
  Novel Sequence
cttcttctccgacgtcaagg

SEQ ID NO: 59 - DNA, 20 bp, Artificial
  Novel Sequence
ccaaatcagtgtgcaaatcg

SEQ ID NO: 60 - DNA, 20 bp, Artificial
  Novel Sequence
tgaatgggtcctgtgtgaaa

SEQ ID NO: 61 - DNA, 20 bp, Artificial
  Novel Sequence
caacggtctgacaacctcct

SEQ ID NO: 62 - DNA, 21 bp, Artificial
  Novel Sequence
ttgctgtgatgtggcattttg

SEQ ID NO: 63 - DNA, 23 bp, Artificial
  Novel Sequence
caggaagcccataaaggcatcaa

SEQ ID NO: 64 - DNA, 20 bp, Artificial
  Novel Sequence
acatcacctgcttcctgacc

SEQ ID NO: 65 - DNA, 20 bp, Artificial
  Novel Sequence
ccagcatcttgatgcagtgt

SEQ ID NO: 66 - DNA, 21 bp, Artificial
  Novel Sequence
ccatctccaaaatcctcagtc

SEQ ID NO: 67 - DNA, 22 bp, Artificial
  Novel Sequence
gctgttaagagcggacaggaaa